SEQUENCE LISTING (77 sequences)

SEQ ID NO: 1 - protein, 17 aa, Artificial Sequence
  peptide derived from Polymorphic membrane protein H of Chlamydia muridarum
SPQVLTPNVIIPFKGDD

SEQ ID NO: 2 - protein, 10 aa, Artificial Sequence
  peptide derived from Nucleoside triphosphatase of Chlamydia muridarum
SMLIIPALGG

SEQ ID NO: 3 - protein, 17 aa, Artificial Sequence
  peptide derived from  D-analyl-D-alanine carboxypeptidase of Chlamydia muridarum
LAAAVMHADSGAILKEK

SEQ ID NO: 4 - protein, 16 aa, Artificial Sequence
  peptide derived from Hypothetical protein of Chlamydia muridarum
DDPEVIRAYIVPPKEP

SEQ ID NO: 5 - protein, 17 aa, Artificial Sequence
  peptide derived from DNA repair protein of Chlamydia muridarum
KIFSPAGLLSAFAKNGA

SEQ ID NO: 6 - protein, 15 aa, Artificial Sequence
  peptide derived from SWIB (YM74) complex protein of Chlamydia muridarum
DPVDMFQMTKIVSKH

SEQ ID NO: 7 - protein, 13 aa, Artificial Sequence
  peptide derived from Translocated actin-recruiting phosphoprotein of Chlamydia muridarum
KLEGIINNNNTPS

SEQ ID NO: 8 - protein, 9 aa, Artificial Sequence
  peptide derived from Exodeoxyribonuclease V, alpha subunit of Chlamydia muridarum
AVPRTSLIF

SEQ ID NO: 9 - protein, 17 aa, Artificial Sequence
  peptide derived from N utilization substance protein A of Chlamydia muridarum
GGAEVILSRSHPEFVKQ

SEQ ID NO: 10 - protein, 8 aa, Artificial Sequence
  peptide derived from Hypothetical protein of Chlamydia muridarum
APILARLS

SEQ ID NO: 11 - protein, 16 aa, Artificial Sequence
  peptide derived from Polymorphic membrane protein F of Chlamydia muridarum
AFHLFASPAANYIHTG

SEQ ID NO: 12 - protein, 19 aa, Artificial Sequence
  peptide derived from Polymorphic membrane protein G of Chlamydia muridarum
NAKTVFLSNVASPIYVDPA

SEQ ID NO: 13 - protein, 17 aa, Artificial Sequence
  peptide derived from Polymorphic membrane protein G of Chlamydia muridarum
ASPIYVDPAAAGGQPPA

SEQ ID NO: 14 - protein, 19 aa, Artificial Sequence
  peptide derived from Ribosomal protein L6 of Chlamydia muridarum
VKGNEVFVSPAAHIIDRPG

SEQ ID NO: 15 - protein, 18 aa, Artificial Sequence
  peptide derived from 3-oxoacyl-(acyl carrier protein) reductase of Chlamydia muridarum
SPGQTNYAAAKAGIIGFS

SEQ ID NO: 16 - protein, 16 aa, Artificial Sequence
  peptide derived from Ani-anti-sigma factor of Chlamydia muridarum
KLDGVSSPAVQESISE

SEQ ID NO: 17 - protein, 15 aa, Artificial Sequence
  peptide derived from ATP dependent Clp protease, proteolytic subunit of Chlamydia muridarum
IGQEITEPLANTVIA

SEQ ID NO: 18 - protein, 15 aa, Artificial Sequence
  peptide derived from Glyceraldehyde 3-phosphate dehydrogenase of Chlamydia muridarum
MTTVHAATATQSVVD

SEQ ID NO: 19 - protein, 14 aa, Artificial Sequence
  peptide derived from Hypothetical protein of Chlamydia muridarum
DLNVTGPKIQTDVD

SEQ ID NO: 20 - protein, 19 aa, Artificial Sequence
  peptide derived from Pyruvate dehydrogenase of Chlamydia muridarum
EGTKIPIGTPIAVFSTEQN

SEQ ID NO: 21 - protein, 17 aa, Artificial Sequence
  peptide derived from Thiol disulfide interchange protein of Chlamydia muridarum
SVPSYVYYPSGNRAPVV

SEQ ID NO: 22 - protein, 15 aa, Artificial Sequence
  peptide derived from Oxidoreductase, DadA family of Chlamydia muridarum
YDHIIVTPGANADIL

SEQ ID NO: 23 - protein, 17 aa, Artificial Sequence
  peptide derived from Metalloprotease, insulinase family of Chlamydia muridarum
LPLMIVSSPKASESGAA

SEQ ID NO: 24 - protein, 16 aa, Artificial Sequence
  peptide derived from Translation elongation factor G of Chlamydia muridarum
GANAIPVHCPIGAESQ

SEQ ID NO: 25 - protein, 15 aa, Artificial Sequence
  peptide derived from Translation elongation factor G of Chlamydia muridarum
VFWLGSKINIIDTPG

SEQ ID NO: 26 - protein, 16 aa, Artificial Sequence
  peptide derived from Translation elongation factor Ts of Chlamydia muridarum
ISRALYTPVNSNQSVG

SEQ ID NO: 27 - protein, 16 aa, Artificial Sequence
  peptide derived from Translation elongation factor Tu of Chlamydia muridarum
FEVQLISPVALEEGMR

SEQ ID NO: 28 - protein, 14 aa, Artificial Sequence
  peptide derived from Translation elongation factor Tu of Chlamydia muridarum
GDAAYIEKVRELMQ

SEQ ID NO: 29 - protein, 15 aa, Artificial Sequence
  peptide derived from Polymorphic membrane protein E of Chlamydia muridarum
SRALYAQPMLAISEA

SEQ ID NO: 30 - protein, 17 aa, Artificial Sequence
  peptide derived from V-type, ATP synthase subunit E of Chlamydia muridarum
KPAEEEAGSIVHNAREQ

SEQ ID NO: 31 - protein, 38 aa, Artificial Sequence
  peptide derived from Yop proteins translocation lipoprotein of Chlamydia trachomatis
KPAPKETPGAAEGAEAQTASEQPSKENAEKQEENNEDA

SEQ ID NO: 32 - protein, 16 aa, Artificial Sequence
  peptide derived from Polymorphic membrane protein E of Chlamydia trachomatis
GSVVFSGATVNSADFH

SEQ ID NO: 33 - protein, 18 aa, Artificial Sequence
  peptide derived from Sigma Regulatory factor of Chlamydia trachomatis
KLDGVSSPAVQESISESL

SEQ ID NO: 34 - protein, 21 aa, Artificial Sequence
  peptide derived from Polymorphic membrane protein H of Chlamydia trachomatis
AEKGGGAIYAPTIDISTNGGS

SEQ ID NO: 35 - protein, 17 aa, Artificial Sequence
  peptide derived from Predicted D-Amino Acid Dehydrogenase of Chlamydia trachomatis
YDHIIVTPGANADILPE

SEQ ID NO: 36 - protein, 16 aa, Artificial Sequence
  peptide derived from Hypothetical protein CT837 of Chlamydia trachomatis
ISYDYSSGNAEASSHN

SEQ ID NO: 37 - protein, 19 aa, Artificial Sequence
  peptide derived from 3-ketoacyl-(acyl-carrier-protein) reductase of Chlamydia trachomatis
GSPGQTNYAAAKAGIIGFS

SEQ ID NO: 38 - protein, 24 aa, Artificial Sequence
  peptide derived from Chaperonin GroEL1 of Chlamydia trachomatis
GPKGRHVVIDKSFGSPQVTKDGVT

SEQ ID NO: 39 - protein, 16 aa, Artificial Sequence
  peptide derived from Hypothetical protein CT144 of Chlamydia trachomatis
GKLIVTNPKSDISFGG

SEQ ID NO: 40 - protein, 20 aa, Artificial Sequence
  peptide derived from Hypothetical protein CT289 of Chlamydia trachomatis
SPKEAAIAAARASLSPEEKR

SEQ ID NO: 41 - protein, 17 aa, Artificial Sequence
  peptide derived from Dihydrolipoamide acetyltransferase of Chlamydia trachomatis
GTKTPIGTPIAVFSTEQ

SEQ ID NO: 42 - protein, 14 aa, Artificial Sequence
  peptide derived from Hypothetical protein CT619 of Chlamydia trachomatis
IPFAKPDANLSAED

SEQ ID NO: 43 - protein, 16 aa, Artificial Sequence
  peptide derived from Type III secretion translocase of Chlamydia trachomatis
ADVLLLSPKASVSPGG

SEQ ID NO: 44 - protein, 18 aa, Artificial Sequence
  peptide derived from Major Outer Membrane Protein of Chlamydia trachomatis
IFDTTTLNPTIAGAGDVK

SEQ ID NO: 45 - protein, 16 aa, Artificial Sequence
  peptide derived from Glyceraldehyde-3-phosphate dehydrogenase of Chlamydia trachomatis
DSTHGSFAPQATFSDG

SEQ ID NO: 46 - protein, 25 aa, Artificial Sequence
  peptide derived from FHA domain; homology to adenylate cyclase of Chlamydia trachomatis
KEGEEDTAESAANEEPKAEASQEEE

SEQ ID NO: 47 - protein, 17 aa, Artificial Sequence
  peptide derived from Clp Protease ATPase of Chlamydia trachomatis
EERVVGQPFAIAAVSDS

SEQ ID NO: 48 - protein, 19 aa, Artificial Sequence
  peptide derived from Muramidase (invasin repeat family) of Chlamydia trachomatis
TPVESTTPVAPEISVVNAK

SEQ ID NO: 49 - protein, 16 aa, Artificial Sequence
  peptide derived from Leucyl aminopeptidase of Chlamydia trachomatis
YKLVYQNALSNFSGKK

SEQ ID NO: 50 - protein, 20 aa, Artificial Sequence
  peptide derived from 50S ribosomal protein L21 of Chlamydia trachomatis
FDGEKASVGAPTVGNAVVKG

SEQ ID NO: 51 - protein, 14 aa, Artificial Sequence
  peptide derived from Hypothetical protein CT143 33 of Chlamydia trachomatis
DLKVTGPTIHTDLD

SEQ ID NO: 52 - protein, 16 aa, Artificial Sequence
  peptide derived from CHLPN 76kDa Homolog of Chlamydia trachomatis
KAPQFGYPAVQNSADS

SEQ ID NO: 53 - protein, 15 aa, Artificial Sequence
  peptide derived from Hypothetical protein CT472 of Chlamydia trachomatis
TPSAVNPLPNPEIDS

SEQ ID NO: 54 - protein, 16 aa, Artificial Sequence
  peptide derived from Polyribonucleotide Nucleotidyltransferase of Chlamydia trachomatis
DAGVPIKAPVAGIAMG

SEQ ID NO: 55 - protein, 14 aa, Artificial Sequence
  peptide derived from Primosome assembly protein of Chlamydia trachomatis
QVFQLITQVTGRSG

SEQ ID NO: 56 - protein, 16 aa, Artificial Sequence
  peptide derived from Polymorphic membrane protein G of Chlamydia trachomatis
AMANEAPIAFIANVAG

SEQ ID NO: 57 - protein, 983 aa, Artificial Sequence
  Polymorphic membrane protein H from Chlamydia muridarum
MLVMPFSLRSTSFCFLACLCSYSYGLASSPQVLTPNVIIPFKGDDIYLNGDCVFASIYAGAEQGSIISANGQNLTIVGQNHTLSFTDSQGPALQNCAFISAEEKISLRDFSSLLFSKNVSCGEKGMISGKTVSISGGDSIVFKDNSVGYSSLPSVGQTPTTPIVGDVLKGSIFCVETGLEISGVKKELVFDNTAGNFGAVFCSRAAQGDTTFTVKDCKGKILFQDNVGSCGGGVIYKGEVLFQDNEGEMLFRGNSAHDDLGILDANPQPPTEVGGGGGVICTPEKTVTFKGNKGPITFDYNFAKGRGGAIQSQTFSLVADSAVVFSNNTAEKGGGAIYALEVNVSTNGGSILFEGNRASEGGAICVSEPIAANNGGLTLHAADGDIIFSKNMTSDRPGERSAIRILDSGTNVSLNASGASKMIFYDPVVQNNPATPPTGTSGEIKINESGSGSVVFTAETLTPSEKLNVINATSNFPGNLTVSSGELVVTKGATLTVGNITATSGRVTLGSGASLSAVAGTAGTCTVSKLGIDLESFLVPTYETAKLGADTTVAVNNNPTLDLVMANETEMYDNPLFMNAVTIPFVTLVSLQTTGGVTTSAVTLNNADTAHYGYQGSWSADWRRPPLAPDPSGMTPLDKSNTLYVTWRPSSNYGVYKLDPQRRGELVPNSLWVSGSALRTFTNGLKEHYVSRDVGFIASVQALGDYVLNYKQGNRDGFLARYGGFQAVAASHYENGGIFGVAFGQLYGQTKSRLYDSKDAGNITILSCFGRSYIDVKGTETVVYWETAYGYSVHRMHTQYFNGKTNKFDHSKCRWHNNSYYAFVGAEHNFLEYCIPTRQLARDYDLTGFMRFEMSGGWSSGAKETGALPRHFDRGTGHNMSLPIGVVAHAVSNGRRSPPSKLTINMGYRPDIWRVTPHCNMKIIANGVKTPIQGSPLARHAFFLEVHDTLYVRHLGRAYMNYSLDARHRQTTHFVSLGLNRIF

SEQ ID NO: 58 - protein, 1016 aa, Artificial Sequence
  Polymorphic membrane protein H from Chlamydia trachomatis
MPFSLRSTSFCFLACLCSYSYGFASSPQVLTPNVTTPFKGDDVYLNGDCAFVNVYAGAENGSIISANGDNLTITGQNHTLSFTDSQGPVLQNYAFISAGETLTLKDFSSLMFSKNVSCGEKGMISGKTVSISGAGEVIFWDNSVGYSPLSIVPASTPTPPAPAPAPAASSSLSPTVSDARKGSIFSVETSLEISGVKKGVMFDNNAGNFGTVFRGNSNNNAGSGGSGSATTPSFTVKNCKGKVSFTDNVASCGGGVVYKGTVLFKDNEGGIFFRGNTAYDDLGILAATSRDQNTETGGGGGVICSPDDSVKFEGNKGSIVFDYNFAKGRGGSILTKEFSLVADDSVVFSNNTAEKGGGAIYAPTIDISTNGGSILFERNRAAEGGAICVSEASSGSTGNLTLSASDGDIVFSGNMTSDRPGERSAARILSDGTTVSLNASGLSKLIFYDPVVQNNSAAGASTPSPSSSSMPGAVTINQSGNGSVIFTAESLTPSEKLQVLNSTSNFPGALTVSGGELVVTEGATLTTGTITATSGRVTLGSGASLSAVAGAANNNYTCTVSKLGIDLESFLTPNYKTAILGADGTVTVNSGSTLDLVMESEAEVYDNPLFVGSLTIPFVTLSSSSASNGVTKNSVTINDADAAHYGYQGSWSADWTKPPLAPDAKGMVPPNTNNTLYLTWRPASNYGEYRLDPQRKGELVPNSLWVAGSALRTFTNGLKEHYVSRDVGFVASLHALGDYILNYTQDDRDGFLARYGGFQATAASHYENGSIFGVAFGQLYGQTKSRMYYSKDAGNMTMLSCFGRSYVDIKGTETVMYWETAYGYSVHRMHTQYFNDKTQKFDHSKCHWHNNNYYAFVGAEHNFLEYCIPTRQFARDYELTGFMRFEMAGGWSSSTRETGSLTRYFARGSGHNMSLPIGIVAHAVSHVRRSPPSKLTLNMGYRPDIWRVTPHCNMEIIANGVKTPIQGSPLARHAFFLEVHDTLYIHHFGRAYMNYSLDARRRQTAHFVSMGLNRIF

SEQ ID NO: 59 - protein, 209 aa, Artificial Sequence
  Nucleoside triphosphatase from Chlamydia muridarum
MKILIASSHGYKVRETKAFLKKIGEFDIFSLVDYPSYTPPKETGETPEENAIQKGVFAAQTFRCWTIADDSMLIIPALGGLPGKLSASFSGEHASDKDHRKKLLEEMLLLENPIDRSAYFECCVVLVSPFGKIFKAHASCEGTIVFKERGSSGFGYDPLFSKHDYKQTYAELPEEIKNQVSHRAKALAKLQPYVEMAFANHLLARNESL

SEQ ID NO: 60 - protein, 209 aa, Artificial Sequence
  Nucleoside triphosphatase from Chlamydia trachomatis
MKILIASSHGYKVRETKVFLKKLGEFDIFSLVDYPSYHPPKETGETPEENAIQKGLFAAQTFRCWTIADDSMLIIPALGGLPGKLSASFAGEQANDKDHRKKLLENMRLLENTIDRSAYFECCVALISPFGKIFKAHASCEGTIAFEERGSSGFGYDPLFVKHDYKQTYAELPEAIKNQVSHRAKALVKLQPYVETVLANHLLAGKESL

SEQ ID NO: 61 - protein, 438 aa, Artificial Sequence
  D-analyl-D-alanine carboxypeptidase from Chlamydia muridarum
MRILSLLCRFFICSSPLFLQPASLLAASPAITTKGLAAAVMHADSGAILKEKNLDQKIFPASMTKIATALLILRKHPDVLTRFITIRREPLTSITPQAKQQSGYRSPPHWLETDGVAIQLKSKEEVSGWDLFHALLISSANDAANVLADACCQSVPAFMHQLNDFLKEIGCQNTHFNSPHGLHHPDHYTTARDLAIIMKEALKEPLFCQVIRTASYTMESTNLSPERTLSSTNRLLSSSSTYFYPPCLGGKTGTTKSAGKNIVFAAEKNNRSIIVVAAGYFGPAAQLYQDAIAVCEDLFNEQLLRCFLLPPASQYSVKTKFGPITAPVSQGIYYDFYPSEGDPLLSLSLESSKIAFPIRQGDLLGHWILSSPSGEKVHSIPFLAESDILPSFKQRILLTSLRILTSYRTYVLILLFFLLNRKKKHSRATKTFSNPFFS

SEQ ID NO: 62 - protein, 343 aa, Artificial Sequence
  D-analyl-D-alanine carboxypeptidase from Chlamydia trachomatis
MRTFFLLYRFFICLAPFFLSFPLYADPHTVLTKGIAAAVVHADSGAILKEKNLDHKIFPASMTKIATALLILRQYPDVLTRFITTRREPLTSITPQAKQQSGYRSPPHWLETDGMTIQLKVKEEVSGWDLFHALLISSANDAANVLADACCQSVSAFMRQLNEFLRELGCQNTHFNSPHGLHHPDHYTTARDLSLIMKEALKEPLFRQVIHTASYTMEATNLSPERVLSSTNKLLSSSSTYFYPPCLGGKTGTTKSAGKNIIFAAEKNNRSIIVVAAGYFGPAAQLYQDAIALCEDLFNEQLLRCFLIPPASHYPVPTRFGTVTAPVAQGIYYDFYPSEEIPS

SEQ ID NO: 63 - protein, 238 aa, Artificial Sequence
  Hypothetical protein from Chlamydia muridarum
MNISGSIKQKLLQFLKKQKSPELLATYLFYLEQSLHLSPVVFVRDKVIFKSAEDAIALLEADKKIWRETEIQISSGKPEVNEQTKRIYICPFTGKVFADNVYANPLDAVYDWLSSCPQNKERQAGVAVKRFLVSDDPEVIRAYIVPPKEPLIKTVYASAITGKLFHSLPTLLEDFKTSYLRPMTLEEVQNQNKFQLESSFLTLLQDALEEEKIAEFVESLADDTAFHEYISQWVDTEE

SEQ ID NO: 64 - protein, 238 aa, Artificial Sequence
  Hypothetical protein from Chlamydia trachomatis
MNISGSIKQKLLQFLKKQKSPELLATYLFYLEQSLHLSPVVFVRDKIIFKSAEDAIQLLEADKKIWRETEIQISSGKPEVNEQTKRIYICPFTGKVFADNVYANPQDAIYDWLSSCPQNRERQSGVAVKRFLVSDDPEVIRAYIVPPKEPIIKTVYASAVTGKLFHSLPTLLEDFKTSYLRPMTLEEVQNQNKFQLESSFLTLLQDALEEEKIAEFVESLADDTAFHKYISQWVDTEE

SEQ ID NO: 65 - protein, 234 aa, Artificial Sequence
  DNA repair protein from Chlamydia muridarum
MQIILPGIVLTHSPAEKQHVIAKIFSPAGLLSAFAKNGASLSCDFRESLLPISFSLFTIQHTPPKMRKVLQGELKNPFTTIKNSYRLLQSTGKMIQAILKTQWQEKPSPQLFSLFLNFLQRIPETPHPYFFSSMFLLKLLQHEGSLDLSHSCTLCKSSLESSTVYRHEGSLFCEKHAHEKTILFSQEEEQILRIIVQAKKFQELMCLAEFPIDIDSKIDSLFSSFLTEKMNVLP

SEQ ID NO: 66 - protein, 243 aa, Artificial Sequence
  DNA repair protein from Chlamydia trachomatis
MQITLPGVVLTNSPAEKQYVIVKIFSPAGLLSAFAKNGASLSCDFRESLFPISFSLFTIQQSPPKMRKVIQGELQNPFTTIKSSYPLLQSAGKMIQAILKTQWHEKPSPHLFSLFLNFLQRIPETQYPNFFSSMFLLKLLQHEGSLDLSRSCTLCKTPLESSTIYRYEGALFCEKHAHEETISFSQEEDHILRVIVQAKKFQELVCLAEFPIDIDTKIDALFSSFLSETSEPSSLYYKGKTLL

SEQ ID NO: 67 - protein, 86 aa, Artificial Sequence
  SWIB (YM74) complex protein from Chlamydia muridarum
MSQNKNSAFMQPVNVSSDLAAIVGTGPMPRTEIIKKIWDYIKQNKLQDPTNKRNINPDDKLAKVFGSKDPVDMFQMTKIVSKHIVK

SEQ ID NO: 68 - protein, 86 aa, Artificial Sequence
  SWIB (YM74) complex protein from Chlamydia trachomatis
MSQNKNSAFMQPVNVSADLAAIVGAGPMPRTEIIKKMWDYIKKNGLQDPTNKRNINPDDKLAKVFGTEKPIDMFQMTKMVSQHIIK

SEQ ID NO: 69 - protein, 1007 aa, Artificial Sequence
  Translocated actin-recruiting phosphoprotein from Chlamydia muridarum
MTTPISNSPSSIPTVTVSTTTASSGSLGTSTVSSTTTSTSVAQTATTTSSASTSIIQSSGENIQSTTGTPSPITSSVSTSAPSPKASATANKTSSAVSGKITSQETSEESETQATTSDGEVSSNYDDVDTPTNSSDSTVDSDYQDVETQYKTISNNGENTYETIGSHGEKNTHVQESHASGTGNPINNQQEAIRQLRSSTYTTSPRNENIFSPGPEGLPNMSLPSYSPTDKSSLLAFLSNPNTKAKMLEHSGHLVFIDTTRSSFIFVPNGNWDQVCSMKVQNGKTKEDLGLKDLEDMCAKFCTGYNKFSSDWGNRVDPLVSSKAGIESGGHLPSSVIINNKFRTCVAYGPWNPKENGPNYTPSAWRRGHRVDFGKIFDGTAPFNKINWGSSPTPGDDGISFSNETIGSEPFATPPSSPSQTPVINVNVNVGGTNVNIGDTNVSKGSGTPTSSQSVDMSTDTSDLDTSDIDTNNQTNGDINTNDNSNNVDGSLSDVDSRVEDDDGVSDTESTNGNDSGKTTSTEENGDPSGPDILAAVRKHLDTVYPGENGGSTEGPLPANQNLGNVIHDVEQNGSAKETIITPGDTGPTDSSSSVDADADVEDTSDTDSGIGDDDGVSDTESTNGNNSGKTTSTEENGDPSGPDILAAVRKHLDTVYPGENGGSTEGPLPANQNLGNVIHDVEQNGAAQETIITPGDTESTDTSSSVNANADLEDVSDADSGFGDDDGISDTESTNGNDSGKNTPVGDGGTPSGPDILAAVRKHLDTVYPGENGGSTERPLPANQNLGDIIHDVEQNGSAKETVVSPYRGGGGNTSSPIGLASLLPATPSTPLMTTPRTNGKAAASSLMIKGGETQAKLVKNGGNIPGETTLAELLPRLRGHLDKVFTSDGKFTNLNGPQLGAIIDQFRKETGSGGIIAHTDSVPGENGTASPLTGSSGEKVSLYDAAKNVTQALTSVTNKVTLAMQGQKLEGIINNNNTPSSIGQNLFAAARATTQSLSSLIGTVQ

SEQ ID NO: 70 - protein, 1005 aa, Artificial Sequence
  Translocated actin-recruiting phosphoprotein from Chlamydia trachomatis
MTNSISGYQPTVTTSTSSTTSASGASGSLGASSVSTTANATVTQTANATNSAATSSIQTTGETVVNYTNSASAPNVTVSTSSSSTQATATSNKTSQAVAGKITSPDTSESSETSSTSSSDHIPSDYDDVGSNSGDISNNYDDVGSNNGDISSNYDDAAADYEPIRTTENIYESIGGSRTSGPENTSGGAAAALNSLRGSSYSNYDDAAADYEPIRTTENIYESIGGSRTSGPENTSGGAAAALNSLRGSSYSNYDDAAADYEPIRTTENIYESIGGSRTSGPENTSDGAAAAALNSLRGSSYTTGPRNEGVFGPGPEGLPDMSLPSYDPTNKTSLLTFLSNPHVKSKMLENSGHFVFIDTDRSSFILVPNGNWDQVCSIKVQNGKTKEDLDIKDLENMCAKFCTGFSKFSGDWDSLVEPMVSAKAGVASGGNLPNTVIINNKFKTCVAYGPWNSQEASSGYTPSAWRRGHRVDFGGIFEKANDFNKINWGTQAGPSSEDDGISFSNETPGAGPAAAPSPTPSSIPIINVNVNVGGTNVNIGDTNVNTTNTTPTTQSTDASTDTSDIDDINTNNQTDDINTTDKDSDGAGGVNGDISETESSSGDDSGSVSSSESDKNASVGNDGPAMKDILSAVRKHLDVVYPGENGGSTEGPLPANQTLGDVISDVENKGSAQDTKLSGNTGAGDDDPTTTAAVGNGAEEITLSDTDSGIGDDVSDTASSSGDESGGVSSPSSESNKNTAVGNDGPSGLDILAAVRKHLDKVYPGDNGGSTEGPLQANQTLGDIVQDMETTGTSQETVVSPWKGSTSSTESAGGSGSVQTLLPSPPPTPSTTTLRTGTGATTTSLMMGGPIKADIITTGGGGRIPGGGTLEKLLPRIRAHLDISFDAQGDLVSTEEPQLGSIVNKFRQETGSRGILAFVESAPGKPGSAQVLTGTGGDKGNLFQAAAAVTQALGNVAGKVNLAIQGQKLSSLVNDDGKGSVGRDLFQAAAQTTQVLSALIDTVG

SEQ ID NO: 71 - protein, 497 aa, Artificial Sequence
  Exodeoxyribonuclease V, alpha subunit from Chlamydia muridarum
MNETLHVQNILQSLLAQHILLPFDLVFAQKHLAQTSSSNREAEAFLVVASALLRCGYPYFTIHKATISPTLPGISNRLLFQWFQALPSDVKATLFEVCDDKIYLRSLFLLREKVFQKLHTLAEAVPRTSLIFENVSQLSEEQNAVLGNVLNSCFSLVCGGPGTGKTFLAVQMIRLILSQIPSAQIVVASPTGKAAAHLHSVLSSQEIIGASVEVVTIHKFLKDVNNGRSPVDLLLIDEGSMVTMNLLHGLIKTIRGEIREGKLFADRMVIFGDVNQLPPIGIGVGNPFHELVSEFTQQAFFLSTSHRAKHKELQELAKSVLHKQPIPFQPLPSRKEAIRRLSDAFVQTAKAGISLCALTPMRQGPWGFLQLNRLLFNEMQEKHPQAPVPIIVTERYETWGLTNGDTGILDPLTQQLHFMNGEILRKEDFPYYSYNYVMSVHKSQGSEYDRVIVILPKGSEVFDSAILYTAITRTKQCVEIWADKETLDMVISKKGRY

SEQ ID NO: 72 - protein, 496 aa, Artificial Sequence
  Exodeoxyribonuclease V, alpha subunit from Chlamydia trachomatis
MNVNQHVQDIVQSLLAQHILLPFDIAFAQKHISQEEVSQEAEAFLATASALLRCGYPYFSICDKTIHPTLPGISSKQLFEWFQVLSSRIKEELFEVVNHKIYLRSLFLLREKVFHKLHRLAGAVPRTPLIFEEIAQLSEEQNQVLKTVLNSCFSLVCGGPGTGKTFLAVQMIRLILAQIPSAQIVVASPTGKASAHLHSVLTSQGIVGDSVEVVTIHKFLKDMRRGCSPVDLLLVDEGSMVTINLLHGLIKTIRGEARGETIYADRMVIFGDANQLPPIGIGVGNPFHEVVSEFSKQACFLSTSHRAKHKELQELASAVLRKELIPFQPLPSRQEAIRRLSFAFTQAAKEGVSLCALTPMRQGLWGFLQLNRLLFNEMQEKHPQAPIPIIVTERYETWGLMNGDTGVLDPVTQQLRFTNGEILHQADFPYYSYNYVMSVHKSQGSEYDRVIVILPKGSEVFDSAILYTAITRTKQHVEIWADREALEAIILKRGRY

SEQ ID NO: 73 - protein, 434 aa, Artificial Sequence
  N utilization substance protein A from Chlamydia muridarum
MNKDLVAIFDYMEREKGIQRSTIVGAIESALKIAAKKTLRDDANVSVSINPRTGDIEVFCEKQIVEKCQNPSKEIPLDKAREYDPDCQIGQYMDVPFVSDQFGRIAAHAARQIIGQKLRHAERDVIYEEYRHRKNEIISGVIKSFSRGSNLVVDLGKVEGLLPARFYPKTEKHKVGDKIYALLYEVQESENGGAEVILSRSHPEFVKQLFMQEVPELEEGSVEIVKIAREAGYRTKMAVRSSSPQTDAVGAFVGMRGSRIKNIIRELNDEKIDVVNYSPVSTELLQNLLYPVEIQKIAILEDDKVIAIIVQDSDYATVIGKRGINARLISQILGYELEVQRMSEYNKLLEIQRLQLAEFEDPRLDQPLEVEGINTLIVQNLEHAGYDTIRKILLASASELASVPGISLELAYKILEQVSKYGACKVDEKPKVED

SEQ ID NO: 74 - protein, 434 aa, Artificial Sequence
  N utilization substance protein A from Chlamydia trachomatis
MNKDLVAIFDYMEREKGIQRSTIVGAIESALKIAAKKTLRDDANVSVSINPRTGDIEVFCEKQIVEKCQNPSKEIPLDKAREYDPDCQIGQYMDVPFISDQFGRIAAHAARQIIGQKLRHAERDVIYEEYRHRKNEIISGVVKSFARGANLVVDLGKVEGLLPARFYPKTEKHKVGDKIYALLYEVQESENGGAEVILSRNHPEFVKQLFVQEVPELEEGSVEIVKIAREAGYRTKMAVRSSNPQTDAVGAFVGMRGSRIKNIIRELNDEKIDVVNYSPVSTELLQNLLYPVEIQKIAILEDDKVIAIIVQDSDYATVIGKRGINARLISQILGYELEVQRVSEYNKLLEIQRLQLAEFEDPRLDQPLEVEGINTLIVQNLEHAGYDTIRKILLASASELASVPGISLELAYKILEQVSKYGEGKVDEKPQVED

SEQ ID NO: 75 - protein, 446 aa, Artificial Sequence
  Hypothetical protein from Chlamydia muridarum
MRTLSISMLILALSCGENTCLCAADSPKAKVDASIGNGASFSPFTGEIKGNRVRLRLAPHTDSSIIKELSKGDCLAVLGESKDYYVVAAPEGVRGYVFRTFVLDNVIEGEKVNVRLEPSTSAPILARLSKGTVVKTLGAAQGKWVEIALPKQCVFYVAKNFVKNVGALELYNQKEGQKKIALDLLNSAMSFADAELQKKVEDIDLDAIYKKMNLAQAEEFKDVPGLQPLVQKALERVQEAFLAKSLEKGSHKTVESYKPVETQAQLQPQRQVIEEKNVSVVPEAPVLSQVEEPKSVLTSSSEVEPLQDVGPIKGSLLSHYIRKKGFVKTSPVVEGRESFERSLFEVWVNLQPEEIRNGLTMESFYRDEQKKKRVLTGELEVYPHIVKNNPGDYLLKNGEDVVAFVYATSIDLSKWLGKRVVLECVSRPNNHFAFPAYIVLSIKEGA

SEQ ID NO: 76 - protein, 433 aa, Artificial Sequence
  Hypothetical protein from Chlamydia trachomatis
MLIFALSFGADACLCAADLSKAKVEASVGDRAAFSPFTGEIKGNRVRLRLAPHTDSFIIKELSKGDCLAVLGESKDYYVVAAPEGVRGYVFRTFVLDNVIEGEKVNVRLEPSTSAPILARLSKGTVVKTLGAAQGKWIEIALPKQCVFYVAKNFVKNVGALDLYNQKEGQKKLALDLLSSAMDFADAELQKKIEDIDLDAIYKKMNLAQSEEFKDVPGLQSLVQKALERVQEAFLAKSLEKSSVKVPEIRHKVLEEIAVVSPAVEETPVVTKTEEQKVTTVPVPAPAVVTEPAQDLSSVKGSLLSHYIRKKGFVKASPVIEGRESFERSLFAVWLSLQPEEIRHQLTMESFYRDEQKKKRVLTGELEVYPHIVKNNPGDYLLKNGEDVVAFVYATSIDLSKWLGKSVVLECVSRPNNHFAFPAYIVLSVKEGA

SEQ ID NO: 77 - protein, 19 aa, Artificial Sequence
  peptide derived from 50S ribosomal protein L6 of Chlamydia trachomatis
VKGNEVFVTPAAHVVDRPG

(19) United States
(12) Patent Application Publication
(10) Pub. No.: US 2015/0010591 A1
(43) Pub. Date: Jan. 8, 2015

(54) CHLAMYDIA ANTIGEN COMPOSITIONS AND USES THEREOF

(71) Applicant: The University of British Columbia, Vancouver (CA)

(72) Inventors: Robert C. Brunham, Sidney (CA); Leonard James Foster, Richmond (CA)

(21) Appl. No.: 14/348,160

(22) PCT Filed: Oct. 1, 2012
(86) PCT No.: PCT/CA2012/050691

§ 371 Date: Mar. 28, 2014

Related U.S. Application Data
(60) Provisional application No. 61/541,944, filed on Sep. 30, 2011.

(51) Int. Cl. A61K 39/118; G01N 33/569

(52) U.S. Cl. CPC A61K 39/118; G01N 33/56927; A61K 2039/55572
USPC 424/190.1; 435/7.24

(57) ABSTRACT

The present invention provides in part peptides and polypeptides derived from Chlamydia app. The present invention also provides in part methods for treating, preventing or diagnosing Chlamydia infection using the peptides and polypeptides.

### STATEMENT REGARDING FEDERALLY-SPONSORED RESEARCH

This research was sponsored at least in part by United States Federal Government Grant No. R01A1076483 from the National Institute Of Allergy and Infectious Diseases (NIAID). The United States Federal Government may have certain rights to the present invention.

### FIELD OF INVENTION

The present invention relates to treatment of bacterial infection. More specifically, the invention provides in part peptides and polypeptides for use against Chlamydia infection.

### BACKGROUND OF THE INVENTION

Chlamydia trachomatis is an intracellular pathogen responsible for over 92 million sexually transmitted infections and 85 million ocular infections per year worldwide (Starnbach, M. N., and N. R. Roan. 2008. Conquering sexually transmitted diseases. Nat Rev Immuno 18:313-317.). Sexually transmitted C. trachomatis is a major cause of long-term disease sequelae in women such as infertility and ectopic pregnancy (Brunham, R. C., D. J. Zhang, X. Yang, and G. M. McClarty. 2000. The potential for vaccine development against chlamydial infection and disease. J Infect Dis 181 Suppl 3:S538-543; Igietseme, J. U., C. M. Black, and H. D. Caldwell. 2002. Chlamydia vaccines: strategies and status. BioDrugs 16:19-35). C. trachomatis infection in women often goes unnoticed until severe reproductive damage (infertility, pelvic inflammatory disease, ectopic pregnancy) is already underway. In addition, women infected with C. trachomatis are at increased risk of contracting HIV following exposure.

The “seek and treat” programs to prevent and control C. trachomatis sexually transmitted infections appear to be failing as case rates and reinfection rates continue to rise (Brunham, R. C., B. Pourbohloul, S. Mak, R. White, and M. L. Rekart. 2005. The unexpected impact of a Chlamydia trachomatis infection control program on susceptibility to reinfection. J Infect Dis 192:1836-1844), possibly due to early treatment interfering with the development of protective immune responses (Su, H., R. Morrison, R. Messer, W. Whitmire, S. Hughes, and H. D. Caldwell. 1999. The effect of doxycycline treatment on the development of protective immunity in a murine model of chlamydial genital infection. J Infect Dis 180:1252-1258).

Previous attempts to vaccinate against C. trachomatis and C. muridarum infection in both human and murine models using dead elementary bodies (EBs), which are non-replicating infectious particles released when infected cells rupture, provided limited protection (Grayston, J. T., and S. P. Wang. 1978. The potential for vaccine against infection of the genital tract with Chlamydia trachomatis. Sex Transm Dis 5:73-77; Grayston, J. T., S. P. Wang, L. J. Yeh, and C. C. Kuo. 1985. Importance of reinfection in the pathogenesis of trachoma. Rev Infect Dis 7:717-725; Lu, H., Z. Xing, and R. C. Brunham. 2002. GM-CSF transgene-based adjuvant allows the establishment of protective mucosal immunity following vaccination with inactivated Chlamydia trachomatis. J Immunol 169:6324-6331; Schachter, J., and H. D. Caldwell. 1980. Chlamydiae. Annu Rev Microbiol 34:285-309). Mice immunized with live C. muridarum EBs have however been shown to generate better protection (Lu, H., Z. Xing, and R. C. Brunham. 2002. GM-CSF transgene-based adjuvant allows the establishment of protective mucosal immunity following vaccination with inactivated Chlamydia trachomatis. J Immunol 169:6324-6331; Su, H., R. Messer, W. Whitmire, E. Fischer, J. C. Portis, and H. D. Caldwell. 1998. Vaccination against chlamydial genital tract infection after immunization with dendritic cells pulsed ex vivo with nonviable Chlamydiae. J Exp Med 188:809-818).

Investigation into the mechanism underlying the efficient induction of immunity provided by live C. muridarum in comparison to dead organisms suggests that dendritic cells (DCs) exposed to live or dead C. muridarum develop into distinct phenotypes. In particular DCs exposed to live C. muridarum become mature and stimulated antigen-specific CD4 T cells, while DCs exposed to dead C. muridarum are inhibited in acquiring a mature phenotype. Co-stimulation of DCs with dead EB and CpG oligodeoxynucleotide has been show to partially overcome dead EB inhibition of DC maturation (Rey-Ladino, J., K. M. Koochesfahani, M. L. Zaharik, C. Shen, and R. C. Brunham. 2005. A live and inactivated Chlamydia trachomatis mouse pneumonitis strain induces the maturation of dendritic cells that are phenotypically and immunologically distinct. Infect Immun 73:1568-1577). Investigation into the transcriptional responses of bone marrow derived DCs following exposure to live and dead C. muridarum using GeneChip microarrays revealed marked differences in CXC chemokine profiles in DCs exposed to live or dead organism (Zaharik, M. L., T. Nayar, R. White, C. Ma, B. A. Vallance, N. Straka, X. Jiang, J. Rey-Ladino, C. Shen, and R. C. Brunham. 2007. Genetic profiling of dendritic cells exposed to live- or ultraviolet-irradiated Chlamydia muridarum reveals marked differences in CXC chemokine profiles. Immunology 120:160-172). In aggregate, the data suggest that DCs exposed to live EBs are phenotypically and functionally distinct from DCs generated by exposure to dead EBs.

Immunity to C. muridarum infection is thought to be largely cell-mediated and therefore dependent on Chlamydia-derived peptides presented to CD4 T cells via MHC molecules on antigen presenting cells (Brunham, R. C., and J. Rey-Ladino. 2005. Immunology of Chlamydia infection: implications for a Chlamydia trachomatis vaccine. Nat Rev Immunol 5:149-161; Steinman, R. M., and M. Pope. 2002. Exploiting dendritic cells to improve vaccine efficacy. J Clin Invest 109:1519-1526; Su, H., and H. D. Caldwell. 1995. CD4+ T cells play a significant role in adoptive immunity to Chlamydia trachomatis infection of the mouse genital tract. Infect Immun 63:3302-3308; Morrison, S. G., H. Su, H. D. Caldwell, and R. P. Morrison. 2000. Immunity to murine Chlamydia trachomatis genital tract reinfection involves B cells and CD4(+) T cells but not CD8(+) T cells. Infect Immun 68:6979-6987; Morrison, R. P., and H. D. Caldwell. 2002. Immunity to murine chlamydial genital infection. Infect Immun 70:2741-2751; Igietseme, J. U., K. H. Ramsey, D. M. Magee, D. M. Williams, T. J. Kincy, and R. G. Rank. 1993. Resolution of murine chlamydial genital infection by the adoptive transfer of a biovar-specific, Th1 lymphocyte clone. Reg Immunol 5:317 -324).

Immunoproteomic approaches (Hunt, D. F., R. A. Henderson, J. Shabanowitz, K. Sakaguchi, H. Michel, N. Sevilir, A. L. Cox, E. Appella, and V. H. Engelhard. 1992. Characterization of peptides bound to the class I MHC molecule HLA-A2.1 by mass spectrometry. Science 255:1261-1263; de Jong, A. 1998. Contribution of mass spectrometry to contemporary immunology. Mass Spectrom Rev 17:311-335; Olsen, J. V., L. M. de Godoy, G. Li, B. Macek, P. Mortensen, R. Pesch, A. Makarov, 0. Lange, S. Horning, and M. Mann. 2005. Parts per million mass accuracy on an Orbitrap mass spectrometer via lock mass injection into a C-trap. Mol Cell Proteomics 4:2010-2021) to identify C. muridarum T cell antigens, based on isolating and sequencing of pathogen-derived peptides binding to MHC class II molecules presented on the surface of DCs after they were pulsed with live EBs, resulted in the identification of a number of C. muridarum peptides derived from 8 novel epitopes (Karunakaran, K. P., J. Rey-Ladino, N. Stoynov, K. Berg, C. Shen, X. Jiang, B. R. Gabel, H. Yu, L. J. Foster, and R. C. Branham. 2008. Immunoproteomic discovery of novel T cell antigens from the obligate intracellular pathogen Chlamydia. J Immunol 180:2459-2465). These peptides were recognized by antigen-specific CD4 T cells in vitro and recombinant proteins containing the MHC binding peptides were able to induce partial protection via to immunization against C. muridarum infection in vivo (Yu, H., X. Jiang, C. Shen, K. P. Karunakaran, and R. C. Branham. 2009. Novel Chlamydia muridarum T cell antigens induce protective immunity against lung and genital tract infection in murine models. J Immunol 182:1602-1608).

Chlamydia sequences (nucleic acid and polypeptide) are described in, for example, U.S. Pat. No. 6,030,799, U.S. Pat. No. 6,696,421, U.S. Pat. No. 6,676,949, U.S. Pat. No. 6,464,979, U.S. Pat. No. 6,653,461, U.S. Pat. No. 6,642,023, U.S. Pat. No. 6,887,843 and U.S. Pat. No. 7,459,524; or in US Patent Publications 2005/0232941, 2009/0022755, and 2008/0102112. Specific Chlamydia antigens are described in, for example, PCT Publication No. WO 2010/085896.

### SUMMARY OF THE INVENTION

The present disclosure provides in part peptides and polypeptides derived from Chlamydia app. The present invention also provides in part methods for treating, preventing or diagnosing Chlamydia infection using the peptides and polypeptides.

In one embodiment, the disclosure provides an immunogenic composition including a polypeptide which includes an amino acid sequence substantially identical to: SPQVLTPNVIIPFKGDD, SMLIIPALGG, LAAAVMHADSGAILKEK, DDPEVIRAYIVPPKEP, KIFSPAGLLSAFAKNGA, DPVDMFQMTKIVSKH, KLEGIINNNNTPS, AVPRTSLIF, GGAEVILSRSHPEFVKQ, APILARLS, or combinations of these polypeptides, together with a physiologically acceptable carrier.

In some embodiments, the polypeptide includes an amino acid sequence substantially identical to: Polymorphic membrane protein H (PmpH), Nucleoside triphosphatase (YggV), D-analyl-D-alanine carboxypeptidase (DacC), a hypothetical protein corresponding to locus tag CT538, DNA repair protein (RecO), SWIB (YM74) complex protein, Translocated actin-recruiting phosphoprotein (Tarp), Exodeoxyribonuclease V, alpha subunit (RecD_{—}2), N utilization substance protein A (NusA), a hypothetical protein corresponding to locus tag CT017, or combinations of these polypeptides, together with a physiologically acceptable carrier.

In alternative embodiments, the composition further includes an additional polypeptide which includes an amino acid sequence substantially identical to: AFHLFASPAANYIHTG, NAKTVFLSNVASPIYVDPA, ASPIYVDPAAAGGQPPA, VKGNEVFVSPAAHIIDRPG, SPGQTNYAAAKAGIIGFS, KLDGVSSPAVQESISE, IGQEITEPLANTVIA, MTTVHAATATQSVVD, DLNVTGPKIQTDVD, EGTKIPIGTPIAVFSTEQN, SVPSYVYYPSGNRAPVV, YDHIIVTPGANADIL, LPLMIVSSPKASESGAA, GANAIPVHCPIGAESQ, VFWLGSKINIIDTPG, ISRALYTPVNSNQSVG, FEVQLISPVALEEGMR, GDAAYIEKVRELMQ, SRALYAQPMLAISEA, or KPAEEEAGSIVHNAREQ, or combinations of these polypeptides.

In some embodiments, the additional polypeptide includes a polypeptide which comprises an amino acid sequence substantially identical to: Polymorphic membrane protein F (PmpF), Polymorphic membrane protein G (PmpG), Ribosomal protein L6 (RplF), 3-oxoacyl-(acyl carrier protein) reductase (FabG), Anti-anti-sigma factor (Aasf), ATP dependent Clp protease, proteolytic subunit (ClpP), Glyceraldehyde 3-phosphate dehydrogenase (Gap), a hypothetical protein corresponding to locus tag CT143, Pyruvate dehydrogenase (PdhC), Thiol disulfide interchange protein (DsbD), Oxidoreductase, DadA family, Metalloprotease, insulinase family, Translation elongation factor G (FusA), Translation elongation factor Ts (Tsf), Translation elongation factor Tu (Tuf), Polymorphic membrane protein E (PmpE), V-type, ATP synthase subunit E (AtpE) , or combinations of these polypeptides.

In some embodiments, the compositions includes PmpG, PmpE, PmpF and PmpH and, optionally, MOMP. In alternative embodiments, the composition includes PmpG, PmpE, PmpF and TC0420 and, optionally, MOMP.

In alternative embodiments, the composition further includes an adjuvant, such as DDA/TDB, DDA/MMG or DDA/MPL.

In some embodiments, the disclosure provides a method for eliciting an immune response against a Chlamydia spp., or component of the Chlamydia spp., in an animal to by administering to the animal an effective amount of the composition described herein, thus eliciting an immune response in the animal. In alternative embodiments, the disclosure provides use of the composition described herein for eliciting an immune response against a Chlamydia spp., or component thereof, in an animal. The immune response may be a cellular immune response.

In some embodiments, the disclosure provides a method for treating or preventing infection by a Chlamydia spp. in an animal by administering to the animal an effective amount of the composition described herein, thus treating or preventing infection by the Chlamydia spp. in the animal. In alternative embodiments, the disclosure provides use of the composition described herein for treating or preventing infection by a Chlamydia spp. in an animal.

In some embodiments, the disclosure provides a method of diagnosing a Chlamydia infection in an animal by determining the presence or absence of a T cell response to a polypeptide which includes an amino acid sequence substantially identical to: SPQVLTPNVIIPFKGDD, SMLIIPALGG, LAAAVMHADSGAILKEK, DDPEVIRAYIVPPKEP, KIFSPAGLLSAFAKNGA, DPVDMFQMTKIVSKH, KLEGIINNNNTPS, AVPRTSLIF, GGAEVILSRSHPEFVKQ, or APILARLS, in a sample from the animal, where the presence of a T cell response indicates a Chlamydia infection in the animal.

In some embodiments, the polypeptide comprises an amino acid sequence substantially identical to: Polymorphic membrane protein H (PmpH), Nucleoside triphosphatase (YggV), D-analyl-D-alanine carboxypeptidase (DacC), a hypothetical protein corresponding to locus tag CT538, DNA repair protein (RecO), SWIB (YM74) complex protein, Translocated actin-recruiting phosphoprotein (Tarp), Exodeoxyribonuclease V, alpha subunit (RecD_{—}2), N utilization substance protein A (NusA), a hypothetical protein corresponding to locus tag CT017.

In alternative embodiments, the sample may be vaginal fluid, vaginal tissue, vaginal washing, vaginal swab, urethral swab, urine, blood, serum, plasma, saliva, semen, urethral discharge, vaginal discharge, ocular fluid, ocular discharge or any combination of these; the animal may be human; the Chlamydia spp. may be a Chlamydia trachomatis or a Chlamydia muridarum.

This summary does not necessarily describe all features of the invention.

### BRIEF DESCRIPTION OF THE DRAWINGS

These and other features of the disclosure will become more apparent from the following description in which reference is made to the appended drawings wherein:

FIG. 1 is a schematic depiction of the sequence of steps involved in the immunoproteomic approach used for Chlamydia T cell vaccine development.

FIG. 2 is a graph showing protective efficacies against Chlamydia genital tract infection in C57 mice vaccinated with different individual Chlamydia proteins formulated with DDA/MPL adjuvant. Cervicovaginal washes were taken at day 6, day 13 and day 20 after infection, and bacterial titers were measured on HeLa 229 cells. *, **, and *** indicate P values of <0.05, <0.01, and <0.001, respectively, in comparison to the PBS group.

FIG. 3 lists amino acid sequences for the polypeptides listed in Table 1.

### DETAILED DESCRIPTION

The present disclosure provides in part peptides and polypeptides derived from Chlamydia app. The present disclosure also provides in part methods for treating, preventing or diagnosing Chlamydia infection using the peptides and polypeptides.

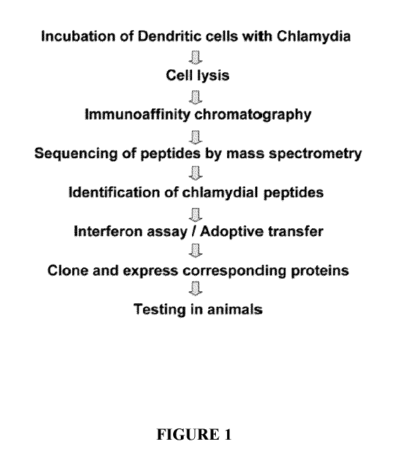

We have identified several new antigens using an immunoproteomic approach as described in FIG. 1. In some embodiments, these antigens may be useful as vaccines or diagnostics for use in the prevention or treatment of Chlamydia spp. infection.

### Chlamydia spp.

By “Chlamydia spp.” is meant a genus of bacteria that are obligate intracellular parasites. Chlamydia spp. include C. trachomatis (a human pathogen) and C. muridarum (pathogenic to mice and hamsters). As C. muridarum and C. trachomatis are highly orthologous pathogenic microbes that have co-evolved with their host species, C. muridarum has been used as a robust animal model for studying cellular immunity and vaccine development.

In some embodiments, a C. trachomatis includes without limitation a C. trachomatis serovar D/UW-3/CX, as well as serovars A, B, Ba, C (implicated in trachoma), serovars D, E, F, G, H, I, J K (implicated in urogenital tract infections) and L1, L2, L3 (lymphogranuloma venereum serovars).

In some embodiments, a C. muridarum includes a C. muridarum mouse pneumonitis (MoPn) strain Nigg.

The genome sequences of various Chlamydia spp. have been determined. The genome sequence of C. trachomatis strain D/UW-3/CX is described for example in Stephens, R. S. et al., 1998 (Genome sequence of an obligate intracellular pathogen of humans: Chlamydia trachomatis. Science 282 (5389): 754-759) and provided in GenBank Accession No. NC_{—}000117.1, GI:15604717; referred to herein as the “the C. trachomatis genome sequence”).

The genome sequence of C. muridarum is described in for example Read, T., et al., 2000 (Genome sequences of Chlamydia trachomatis MoPn and Chlamydia pneumoniae AR39 Nucleic Acids Res. 28 (6): 1397-1406) and provided in GenBank Accession No. NC_{—}002620.2, GI:29337300; referred to herein as the “the C. muridarum genome sequence”).

### Chlamydia spp. Polypeptides and Nucleic Acid Molecules

Compounds for use in the compositions and methods according to the disclosure include, without limitation, the peptides or polypeptides described herein, for example, those listed in Tables 1-4, as well as nucleic acid molecules encoding these peptides or polypeptides.

In some embodiments, compounds for use in the compositions and methods according to the disclosure include, without limitation, a C. muridarum or C. trachomatis sequence such as an amino acid sequence substantially identical to one or more of the sequences listed in Tables 1-4.

In some embodiments, compounds for use in the compositions and methods according to the disclosure include, without limitation, a C. muridarum or C. trachomatis sequence such as a nucleic acid sequence that encodes an amino acid sequence substantially identical to one or more of the sequences listed in Tables 1-4.

In alternative embodiments, compounds for use in the compositions and methods according to the disclosure include, without limitation, one or more of the peptides or polypeptides as described in Table 1.

In alternative embodiments, compounds for use in the compositions and methods according to the disclosure include, without limitation, one or more of peptides including the following amino acid sequences: SPQVLTPNVIIPFKGDD, SMLIIPALGG, LAAAVMHADSGAILKEK, DDPEVIRAYIVPPKEP, KIFSPAGLLSAFAKNGA, DPVDMFQMTKIVSKH, KLEGIINNNNTPS, AVPRTSLIF, GGAEVILSRSHPEFVKQ, or APILARLS (SEQ ID NOs.: 1-10).

In alternative embodiments, compounds for use in the compositions and methods according to the disclosure include, without limitation, one or more of the peptides or polypeptides described in Table 1 in combination with one or more of the peptides or polypeptides described in Table 2.

In alternative embodiments, compounds for use in the compositions and methods according to the disclosure include, without limitation, one or more of the peptides or polypeptides described in Table 1 in combination with one or more of the peptides or polypeptides described in Tables 3 or 4.

In alternative embodiments, compounds for use in the compositions and methods according to the disclosure further include, without limitation, one or more of a C. trachomatis polypeptide such as amino acid permease (gi:3328837), Ribosomal protein L6 (RpIF, gi:3328951), 3-oxoacyl-(acyl carrier protein) reductase (FabG, gi: 15604958), Anti anti sigma factor (Aasf, gi: 15605151), Polymorphic membrane protein G (PmpG, gi:3329346), Hypothetical protein (TC0420, gi: 15604862), ATP dependent CIp protease (Clpl, gi: 15605439), Polymorphic membrane protein F ( PmpF, gi:3329345), Glyceraldehyde 3-phosphate dehydrogenase (Gap, gi: 15605234) and major outer membrane protein 1 (MOMP) (gi:3329133), or fragments or portions thereof. Examples of fragments or portions of the above-referenced polypeptides include amino acids 25-512 of PmpG (PmpG₂₅₋₅₁₂), amino acids 26-585 of PmpF (PmpF₂₆₋₅₈₅), and amino acids 22-393 of MOMP.

In alternative embodiments, compounds for use in the compositions and methods according to the disclosure further include, without limitation, one or more of a C. muridarum polypeptide such as amino acid permease (gi: 15835268), Ribosomal protein L6 (RpIF, gi: 15835415), 3_oxoacyl (acyl carrier protein) reductase (FabG, gi: 15835126), Anti anti sigma factor (Aasf, gi: 15835322), Polymorphic membrane protein G (PmpG or PmpG-1, gi: 15834883), Hypothetical protein TC0420(gi: 15835038), ATP dependent CIp protease_proteolytic subunit (CIp, gi: 15834704), Polymorphic membrane protein F (PmpF or PmpE/F, gi: 15834882), Glyceraldehyde 3_phosphate dehydrogenase (Gap, gi: 15835406) and major outer membrane protein 1 (MOMP, gi7190091), or fragments or portions thereof. Examples of fragments or portions of the above-referenced polypeptides include amino acids 25-500 of PmpG-1 (PmpG-1₂₅₋₅₀₀), amino acids 25-575 of PmpE/F-2 (PmpE/F-2₂₅₋₅₇₅), and amino acids 23-387 of MOMP.

In some embodiments, compounds for use in the compositions and methods according to the disclosure include, without limitation, peptides or polyeptides from a combination of two or more of PmpG, PmpF, PmpE, PmpH, Rpl F, Aasf, RecO, Tarp, AtpE, TC0420, TC0190, TC0825 or TC0285, as long as at least one of the polypeptides is PmpH, RecO, Tarp, AtpE, TC0190, TC0825 or TC0285 or an immunogenic fragment thereof.

In some embodiments, compounds for use in the compositions and methods according to the disclosure include, without limitation, peptides or polypeptides from a combination of two or more of PmpE, Sigma regulatory factor (RsbV), 50S ribosomal protein L6 (R16), PmpH, predicted D-amino acid dehydrogenase, 3-ketoacyl-(acyl-carrier-protein) reductase (FabG), Dihydrolipoamide acetyltransferase (PdhC), glyceraldehyde-3-phosphate dehydrogenase (GapA), hypothetical protein CT143 and PmpG, as long as at least one of the polypeptides is PmpH, or an immunogenic fragment thereof.

In some embodiments, compounds for use in the compositions and methods according to the disclosure include, without limitation, peptides or polypeptides from a combination of two or more of metalloprotease (insulinase family), PmpE, AtpE, PmpH, TC0825, RecO, SWIB (YM74) complex protein and TC0285, as long as at least one of the polypeptides is PmpH, RecO, AtpE, or TC0825 or an immunogenic fragment thereof.

In some embodiments, compounds for use in the compositions and methods according to the disclosure include, without limitation, peptides or polypeptides from a combination of PmpG, PmpE, PmpF and PmpH and, optionally, MOMP.

In some embodiments, compounds for use in the compositions and methods according to the disclosure include, without limitation, peptides or polypeptides from a combination of PmpG, PmpE, PmpF and TC0420 and, optionally, MOMP.

In general, it is to be understood that the sequences of polypeptides and amino acids referenced herein correspond to those indicated in the locus tags referenced in the C. trachomatis genome sequence and/or the C. muridarum genome sequence.

In some embodiments, compositions for use according to the disclosure include multiple peptides and/or polypeptides, for example, at least 2, 3, 4, 5, 6, 7, 8, 9, 10, 11, 12, 13, 14, 15, 16, or more.

It is well known in the art that some modifications and changes can be made in the structure of a polypeptide without substantially altering the biological function of that peptide, to obtain a biologically equivalent polypeptide. Accordingly, it will be appreciated by a person of skill in the art that the numerical designations of the positions of amino acids within a sequence are relative to the specific sequence. Also the same positions may be assigned different numerical designations depending on the way in which the sequence is numbered and the sequence chosen. Furthermore, sequence variations such as insertions or deletions, may change the relative position and subsequently the numerical designations of particular amino acids at and around a to site.

In some embodiments, the peptides or polypeptides may be provided in combination with a heterologous peptides or polypeptide, such as an epitope tag.

A “protein,” “peptide” or “polypeptide” is any chain of two or more amino acids, including naturally occurring or non-naturally occurring amino acids or amino acid analogues, regardless of post-translational modification (e.g., glycosylation or phosphorylation). An “amino acid sequence”, “polypeptide”, “peptide” or “protein” of the invention may include peptides or proteins that have abnormal linkages, cross links and end caps, non-peptidyl bonds or alternative modifying groups. Such modified peptides are also within the scope of the invention. The term “modifying group” is intended to include structures that are directly attached to the peptidic structure (e.g., by covalent coupling), as well as those that are indirectly attached to the peptidic structure (e.g., by a stable non-covalent association or by covalent coupling to additional amino acid residues, or mimetics, analogues or derivatives thereof, which may flank the core peptidic structure). For example, the modifying group can be coupled to the amino-terminus or carboxy-terminus of a peptidic structure, or to a peptidic or peptidomimetic region flanking the core domain.

Alternatively, the modifying group can be coupled to a side chain of at least one amino acid residue of a peptidic structure, or to a peptidic or peptido-mimetic region flanking the core domain (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain). Modifying groups covalently coupled to the peptidic structure can be attached by means and using methods well known in the art for linking chemical structures, including, for example, amide, alkylamino, carbamate or urea bonds.

In one aspect of the invention, polypeptides of the present invention also extend to biologically equivalent peptides or “variants' that differ from a portion of the sequence of the polypeptides of the present invention by conservative amino acid substitutions, or differ by non-conservative substitutions that do not affect biological function e.g., immunogenicity. As used herein, the term “conserved amino acid substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

As used herein, the term “amino acids” means those L-amino acids commonly found in naturally occurring proteins, D-amino acids and such amino acids when they have been modified. Accordingly, amino acids of the invention may include, for example: 2-Aminoadipic acid; 3-Aminoadipic acid; beta-Alanine; beta-Aminopropionic acid; 2-Aminobutyric acid; 4-Aminobutyric acid; piperidinic acid; 6-Aminocaproic acid; 2-Aminoheptanoic acid; 2-Aminoisobutyric acid; 3-Aminoisobutyric acid; 2-Aminopimelic acid; 2,4 Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; sarcosine; N-Methylisoleucine; 6-N-methyllysine; N-Methylvaline; Norvaline; Norleucine; and Ornithine.

In some embodiments, conserved amino acid substitutions may be made where an amino acid residue is substituted for another having a similar hydrophilicity value (e.g., within a value of plus or minus 2.0, or plus or minus 1.5, or plus or minus 1.0, or plus or minus 0.5), where the following may be an amino acid having a hydropathic index of about −1.6 such as Tyr (−1.3) or Pro (−1.6) are assigned to amino acid residues (as detailed in U.S. Pat. No. 4,554,101, incorporated herein by reference): Arg (+3.0); Lys (+3.0); Asp (+3.0); Glu (+3.0); Ser (+0.3); Asn (+0.2); Gin (+0.2); Gly (0); Pro (−0.5); Thr (−0.4); Ala (−0.5); His (−0.5); Cys (−1.0); Met (−1.3); Val (−1.5); Leu (−1.8); lie (−1.8); Tyr (−2.3); Phe (−2.5); and Trp (−3.4).

In alternative embodiments, conservative amino acid substitutions may be made where an amino acid residue is substituted for another having a similar hydropathic index (e.g., within a value of plus or minus 2.0, or plus or minus 1.5, or plus or minus 1.0, or plus or minus 0.5). In such embodiments, each amino acid residue may be assigned a hydropathic index on the basis of its hydrophobicity and charge characteristics, as follows: He (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glu (−3.5); Gin (−3.5); Asp (−3.5); Asn (−3.5); Lys (−3.9); and Arg (−4.5).

In alternative embodiments, conservative amino acid substitutions may be made using publicly available families of similarity matrices (60, 70, 102, 103, 94, 104, 86) The PAM matrix is based upon counts derived from an evolutionary model, while the Blosum matrix uses counts derived from highly conserved blocks within an alignment.

A similarity score of above zero in either of the PAM or Blosum matrices may be used to make conservative amino acid substitutions.

In alternative embodiments, conservative amino acid substitutions may be made where an amino acid residue is substituted for another in the same class, where the amino acids are divided into non-polar, acidic, basic and neutral classes, as follows: non-polar: Ala, Val, Leu, He, Phe, Trp, Pro, Met; acidic: Asp, Glu; basic: Lys, Arg, His; neutral: Gly, Ser, Thr, Cys, Asn, Gln, Tyr.

Conservative amino acid changes can include the substitution of an L-amino acid by the corresponding D-amino acid, by a conservative D-amino acid, or by a naturally-occurring, non-genetically encoded form of amino acid, as well as a conservative substitution of an L-amino acid. Naturally-occurring non-genetically encoded amino acids include beta-alanine, 3-amino-propionic acid, 2,3-diamino propionic acid, alpha-aminoisobutyric acid, 4-amino-butyric acid, N-methylglycine (sarcosine), hydroxyproline, ornithine, citrulline, t-butylalanine, t-butylglycine, N-methylisoleucine, phenylglycine, cyclohexylalanine, norleucine, norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, homoarginine, N-acetyl lysine, 2-amino butyric acid, 2-amino butyric acid, 2,4,-diamino butyric acid, p-aminophenylalanine, N-methylvaline, homocysteine, homoserine, cysteic acid, epsilon-amino hexanoic acid, delta-amino valeric acid, or 2,3-diaminobutyric acid.

In alternative embodiments, conservative amino acid changes include changes based on considerations of hydrophilicity or hydrophobicity, size or volume, or charge. Amino acids can be generally characterized as hydrophobic or hydrophilic, depending primarily on the properties of the amino acid side chain. A hydrophobic amino acid exhibits a hydrophobicity of greater than zero, and a hydrophilic amino acid exhibits a hydrophilicity of less than zero, based on the normalized consensus hydrophobicity scale of Eisenberg et al. (Ann. Rev. Biochem. 53: 595-623, 1984). Genetically encoded hydrophobic amino acids include Gly, Ala, Phe, Val, Leu, He, Pro, Met and Trp, and genetically encoded hydrophilic amino acids include Thr, His, Glu, Gln, Asp, Arg, Ser, and Lys. Non-genetically encoded hydrophobic amino acids include t-butylalanine, while non-genetically encoded hydrophilic amino acids include citrulline and homocysteine.

Hydrophobic or hydrophilic amino acids can be further subdivided based on the characteristics of their side chains. For example, an aromatic amino acid is a hydrophobic amino acid with a side chain containing at least one aromatic or heteroaromatic ring, which may contain one or more substituents such as —OH, —SH, —CN, —F, —CI, —Br, —I, —NO₂, —NO, —NH₂, —NHR, —NRR, —C(O)R, —C(O)OH, —C(O)OR, —C(O)NH₂, —C(O)NHR, —C(O)NRR, etc., where R is independently (—C₆) alkyl, substituted (C₁-C₆) alkyl, (C C₆) alkenyl, substituted (-C₆) alkenyl, (Cj-C₆) alkynyl, substituted (C_{Ï}-C₆) alkynyl, (C₅-C₂o) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl or substituted 6-26 membered alkheteroaryl. Genetically encoded aromatic amino acids include Phe, Tyr, and Trp, while non-genetically encoded aromatic amino acids include phenylglycine, 2-napthylalanine, beta-2-thienylalanine, 1 ,2,3, 4-tetrahydro-isoquinoline-3 -carboxylic acid, 4-chlorophenylalanine, 2-fluorophenylalanine3-fluorophenylalanine, and 4-fluorophenylalanine.

An apolar amino acid is a hydrophobic amino acid with a side chain that is uncharged at physiological pH and which has bonds in which a pair of electrons shared in common by two atoms is generally held equally by each of the two atoms (i.e., the side chain is not polar). Genetically encoded apolar amino acids include Gly, Leu, Val, He, Ala, and Met, while non-genetically encoded apolar amino acids include cyclohexylalanine. Apolar amino acids can be further subdivided to include aliphatic amino acids, which is a hydrophobic amino acid having an aliphatic hydrocarbon side chain. Genetically encoded aliphatic amino acids include Ala, Leu, Val, and He, while non-genetically encoded aliphatic amino acids include norleucine.

A polar amino acid is a hydrophilic amino acid with a side chain that is uncharged at physiological pH, but which has one bond in which the pair of electrons shared in common by two atoms is held more closely by one of the atoms. Genetically encoded polar amino acids include Ser, Thr, Asn, and Gin, while non-genetically encoded polar amino acids include citrulline, N-acetyl lysine, and methionine sulfoxide.

An acidic amino acid is a hydrophilic amino acid with a side chain pKa value of less than 7. Acidic amino acids typically have negatively charged side chains at physiological pH due to loss of a hydrogen ion. Genetically encoded acidic amino acids include Asp and Glu. A basic amino acid is a hydrophilic amino acid with a side chain pKa value of greater than 7. Basic amino acids typically have positively charged side chains at physiological pH due to association with hydronium ion. Genetically encoded basic amino acids include Arg, Lys, and His, while non-genetically encoded basic amino acids include the non-cyclic amino acids ornithine, 2,3,-diaminopropionic acid, 2,4-diaminobutyric acid, and homoarginine. It will be appreciated by one skilled in the art that the above classifications are not absolute and that an amino acid may be classified in more than one category. In addition, amino acids can be classified based on known behaviour and or characteristic chemical, physical, or biological properties based on specified assays or as compared with previously identified amino acids. Amino acids can also include bifunctional moieties having amino acid-like side chains.

Conservative changes can also include the substitution of a chemically derivatised moiety for a non-derivatised residue, by for example, reaction of a functional side group of an amino acid. Thus, these substitutions can include compounds whose free amino groups have been derivatised to amine hydrochlorides, p-toluene sulfonyl groups, carbobenzoxy groups, t-butyloxycarbonyl groups, chloroacetyl groups or formyl groups. Similarly, free carboxyl groups can be derivatized to form salts, methyl and ethyl esters or other types of esters or hydrazides, and side chains can be derivatized to form O-acyl or O-alkyl derivatives for free hydroxyl groups or N-imbenzylhistidine for the imidazole nitrogen of histidine.

Peptides or peptide analogues can be synthesised by standard chemical techniques, for example, by automated synthesis using solution or solid phase synthesis methodology. Automated peptide synthesisers are commercially available and use techniques well known in the art. Peptides and peptide analogues can also be prepared using recombinant DNA technology using standard methods such as those described in, for example, Sambrook, et al. (Molecular Cloning: A Laboratory Manual. 3r^{d} ed., Cold Spring Harbor Laboratory, Cold Spring Harbor Laboratory Press, Cold Spring Harbor, N.Y., 2000) or Ausubel et al. (Current Protocols in Molecular Biology, John Wiley & Sons, New York, N.Y., 1987-2012).

Accordingly, and as discussed herein, compounds for use according to the disclosure include nucleic acid molecules encoding the peptides or polypeptides disclosed herein.

The terms “nucleic acid” or “nucleic acid molecule” encompass both RNA (plus and minus strands) and DNA, including cDNA, genomic DNA, and synthetic (e.g., chemically synthesized) DNA. The nucleic acid may be double-stranded or single-stranded. Where single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. One example of a modified RNA included within this term is phosphorothioate RNA. By “DNA” is meant a sequence of two or more covalently bonded, naturally occurring or modified deoxyribonucleotides. By “cDNA” is meant complementary or copy DNA produced from an RNA template by the action of RNA-dependent DNA polymerase (reverse transcriptase). Thus a “cDNA clone” means a duplex DNA sequence complementary to an RNA molecule of interest, carried in a cloning vector. By “complementary” is meant that two nucleic acids, e.g., DNA or RNA, contain a sufficient number of nucleotides which are capable of forming Watson-Crick base pairs to produce a region of double-strandedness between the two nucleic acids. Thus, adenine in one strand of DNA or RNA pairs with thymine in an opposing complementary DNA strand or with uracil in an opposing complementary RNA strand. It will be understood that each nucleotide in a nucleic acid molecule need not form a matched Watson-Crick base pair with a nucleotide in an opposing complementary strand to form a duplex. A nucleic acid molecule is “complementary” to another nucleic acid molecule if it hybridizes, under conditions of high stringency, with the second nucleic acid molecule.

A compound is “isolated” when it is separated from the components that naturally accompany it. Typically, a compound is isolated when it is at least 10%, 20%, 30%, 40%, 50%, or 60%, or more generally at least 70%, 75%, 80%, 85%, 90%, 95%, or 99% by weight, of the total material in a sample. Thus, for example, a polypeptide that is chemically synthesized or produced by recombinant technology will be generally be substantially free from its naturally associated components. A nucleic acid molecule will generally be substantially pure or “isolated” when it is not immediately contiguous with (i.e., covalently linked to) the coding sequences with which it is normally contiguous in the naturally occurring genome of the organism from which the DNA of the invention is derived. Therefore, an “isolated” gene or nucleic acid molecule is intended to mean a gene or nucleic acid molecule which is not flanked by nucleic acid molecules which normally (in nature) flank the gene or nucleic acid molecule (such as in genomic sequences) and/or has been completely or partially purified from other transcribed sequences (as in a cDNA or RNA library). For example, an isolated nucleic acid of the invention may be substantially isolated with respect to the complex cellular milieu in which it naturally occurs. The term therefore includes, e.g., a recombinant nucleic acid incorporated into a vector, such as an autonomously replicating plasmid or virus; or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant nucleic acid which is part of a hybrid gene encoding additional polypeptide sequences. Preferably, an isolated nucleic acid comprises at least about 50, 80 or 90 percent (on a molar basis) of all macromolecular species present. Thus, an isolated gene or nucleic acid molecule can include a gene or nucleic acid molecule which is synthesized chemically or by recombinant means. Recombinant DNA contained in a vector are included in the definition of “isolated” as used herein. Also, isolated nucleic acid molecules include recombinant DNA molecules in heterologous host cells, as well as partially or substantially purified DNA molecules in solution. In vivo and in vitro RNA transcripts of the DNA molecules of the present invention are also encompassed by “isolated” nucleic acid molecules.

Various genes and nucleic acid sequences of the invention may be to recombinant sequences. The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. The term “recombinant” when made in reference to genetic composition refers to a gamete or progeny with new combinations of alleles that did not occur in the parental genomes Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as ‘recombinant’ therefore indicates that the nucleic acid molecule has been manipulated using genetic engineering, i.e. by human intervention.

Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

As used herein, “heterologous” in reference to a nucleic acid or protein is a molecule that has been manipulated by human intervention so that it is located in a place other than the place in which it is naturally found. For example, a nucleic acid sequence from one species may be introduced into the genome of another species, or a nucleic acid sequence from one genomic locus may be moved to another genomic or extrachromasomal locus in the same species. A heterologous protein includes, for example, a protein expressed from a heterologous coding sequence or a protein expressed from a recombinant gene in a cell that would not naturally express the protein.

A “substantially identical” sequence is an amino acid or nucleotide sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a sequence can be any integer from 10% to 99%, or more generally at least 10%, 20%, 30%, 40%, 50, 55% or 60%, or at least 65%, 75%, 80%, 85%, 90%, or 95%, or as much as 96%, 97%, 98%, or 99% identical at the amino acid or nucleotide level to the sequence used for comparison using, for example, the Align Program (96) or FASTA. For polypeptides, the length of comparison sequences may be at least 2, 5, 10, or 15 amino acids, or at least 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various substitutions, deletions, substitutions, and other modifications.

Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8x SSC, 0.2 M Tris-Cl, pH 7.6, lx Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology (Ausubel et al, Current Protocols in Molecular Biology, John Wiley & Sons, New York, N.Y., 1998).

Substantially identical sequences may for example be sequences that are substantially identical to the Chlamydia spp. sequences described or referenced herein. A substantially identical sequence may for example be an amino acid sequence that is substantially identical to the sequence of any one of SEQ ID NOs: 1-76, or to any one of the sequences indicated by the locus tags referenced in the C. trachomatis genome sequence and/or the C. muridarum genome sequence as indicated herein, or a fragment or variant thereof, or a nucleotide sequence substantially identical to the sequence of any one of SEQ ID NOs: of SEQ ID NOs: 1-76, or to any one of the sequences indicated by the locus tags referenced in the C. trachomatis genome sequence and/or the C. muridarum genome sequence as indicated herein, or a fragment or variant thereof. In some embodiments, a substantially identical sequence may for example be a nucleotide sequence that is complementary to or hybridizes with the sequence of any one of SEQ ID NOs: 1-76, or to any one of the sequences indicated by the locus tags referenced in the C. trachomatis genome sequence and/or the C. muridarum genome sequence as indicated herein, or a fragment or variant thereof. In some embodiments, a substantially identical sequence may be derived from a Chlamydia spp., such as a C. trachomatis or a C. muridarum.

### Pharmaceutical & Veterinary Compositions, Dosages, and Administration

The compounds and compositions as described herein may be used to prepare vaccine or other formulations. The compounds and compositions can be provided alone or in combination with other compounds (for example, nucleic acid molecules, small molecules, polyp eptides, peptides, or peptide analogues), in the presence of a liposome, an adjuvant, or any pharmaceutically acceptable carrier, in a form suitable for administration to an animal subject, for example, mice, humans, pigs, etc. If desired, treatment with a compound according to the invention may be combined with more traditional and existing therapies for Chlamydia infection.

Conventional pharmaceutical practice may be employed to provide suitable formulations to administer the compounds or compositions to subjects infected by a Chlamydia pathogen. Any appropriate route of administration may be employed, for example, parenteral, intravenous, subcutaneous, intramuscular, intracranial, intrathecal, intraorbital, ophthalmic, intraventricular, intracapsular, intraspinal, intracisternal, intraperitoneal, intranasal, epidermal, transdermal, mucosal membrane aerosol, nasal, rectal, vaginal, topical or oral administration. In some embodiments, the compounds or compositions described herein may be applied to epithelial surfaces. Some epithelial surfaces may comprise a mucosal membrane, for example buccal, gingival, nasal, tracheal, bronchial, gastrointestinal, rectal, urethral, vaginal, cervical, uterine and the like. Some epithelial surfaces may comprise keratinized cells, for example, skin, tongue, gingival, palate or the like.

Formulations may be in the form of liquid solutions or suspensions; tablets or capsules; powders, nasal drops, or aerosols. Methods are well known in the art for making formulations (Berge et al. 1977. J. Pharm Sci. 66: 1-19); Remington—The Science and Practice of Pharmacy, 21⁴ edition. Gennaro et al editors. Lippincott Williams & Wilkins Philadelphia.). Such excipients may include, for example, salts, buffers, antioxidants, complexing agents, tonicity agents, cryoprotectants, lyoprotectants, suspending agents, emulsifying agents, antimicrobial agents, preservatives, chelating agents, binding agents, surfactants, wetting agents, anti-adherents agents, disentegrants, coatings, glidants, deflocculating agents, anti-nucleating agents, surfactants, stabilizing agents, non-aqueous vehicles such as fixed oils, polymers or encapsulants for sustained or controlled release, ointment bases, fatty acids, cream bases, emollients, emulsifiers, thickeners, preservatives, solubilizing agents, humectants, water, alcohols or the like.

Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds or compositions.

Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

For therapeutic or prophylactic compositions, the compounds or compositions are administered to an animal in an amount effective to stop or slow a Chlamydia infection.

An “effective amount” of a compound according to the invention includes a therapeutically effective amount or a prophylactically effective amount. A “therapeutically effective amount” refers to an amount effective, at dosages and for periods of time necessary, to achieve the desired therapeutic result, such as reduction of a Chlamydia infection or induction of an immune response to a Chlamydia antigen or epitope. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as prevention of a Chlamydia infection or induction of an immune response to a Chlamydia antigen or epitope. Typically, a prophylactic dose is used in subjects prior to or at an earlier stage of disease, so that a prophylactically effective amount may be less than a therapeutically effective amount. A suitable range for therapeutically or prophylactically effective amounts of a compound maybe any integer from 0.1 nM-0.1 M, 0.1 nM-0.05 M, 0.05 nM-15 μM or 0.01 nM-10 μM.

In some embodiments, an effective amount may be calculated on a mass/mass basis (e.g. micrograms or milligrams per kilogram of subject), or may be calculated on a mass/volume basis (e.g. concentration, micrograms or milligrams per milliliter). Using a mass/volume unit, one or more peptides or polypeptides may be present at an amount from about 0.1 ug/ml to about 20 mg/ml, or any amount therebetween, for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/ml, or any amount therebetween; or from about 1 ug/ml to about 2000 ug/ml, or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, ug/ml or any amount therebetween; or from about 10 ug/ml to about 1000 ug/ml or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/ml, or any amount therebetween; or from about 30 ug/ml to about 1000ug/ml or any amount therebetween, for example 30.0, 35.0, 40.0, 50.0 60.0, 70.0, 80.0, 90.0, 100, 120, 140, 160 180, 200, 250, 500, 750, 1000 ug/ml.

Quantities and/or concentrations may be calculated on a mass/mass basis (e.g. micrograms or milligrams per kilogram of subject), or may be calculated on a mass/volume basis (e.g. concentration, micrograms or milligrams per milliliter). Using a mass/volume unit, one or more peptides or polypeptides may be present at an amount from about 0.1 ug/ml to about 20 mg/ml, or any amount therebetween, for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/ml, or any amount therebetween; or from about 1 ug/ml to about 2000 ug/ml, or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, ug/ml or any amount therebetween; or from about 10 ug/ml to about 1000ug/ml or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/ml, or any amount therebetween; or from about 30 ug/ml to about 1000 ug/ml or any amount therebetween, for example 30.0, 35.0, 40.0, 50.0 60.0, 70.0, 80.0, 90.0, 100, 120, 140, 160 180, 200, 250, 500, 750, 1000 ug/ml.

Compositions according to various embodiments of the invention, including therapeutic compositions, may be administered as a dose comprising an effective amount of one or more peptides or polypeptides. The dose may comprise from about 0.1 ug/kg to about 20 mg/kg (based on the mass of the subject), for example 0.1, 0.5, 1, 2, 5, 10, 15, 20, 25, 30, 35, 40, 50, 60, 70, 80, 90, 100, 120, 140, 160 180, 200, 250, 500, 750, 1000, 1500, 2000, 5000, 10000, 20000 ug/kg, or any amount therebetween; or from about lug/kg to about 2000ug/kg or any amount therebetween, for example 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000 ug/kg, or any amount therebetween; or from about 10 ug/kg to about 1000 ug/kg or any amount therebetween, for example 10.0, 15.0, 20.0, 25.0, 30.0, 35.0, 40.0, 50.0 60.0, 70.0, 80.0, 90.0, 100, 120, 140, 160 180, 200, 250, 500, 750, 1000 ug/kg, or any amount therebetween; or from about 30 ug/kg to about 1000ug/kg or any amount therebetween, for example 30.0, 35.0, 40.0, 50.0 60.0, 70.0, 80.0, 90.0, 100, 120, 140, 160 180, 200, 250, 500, 750, 1000 ug/kg.

One of skill in the art will be readily able to interconvert the units as necessary, given the mass of the subject, the concentration of the composition, individual components or combinations thereof, or volume of the composition, individual components or combinations thereof, into a format suitable for the desired application.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

The amount of a composition administered, where it is administered, the method of administration and the timeframe over which it is administered may all contribute to the observed effect. As an example, a composition may be administered systemically e.g. intravenous administration and have a toxic or undesirable effect, while the same composition administered subcutaneously or intranasally may not yield the same undesirable effect. In some embodiments, localized stimulation of immune cells in the lymph nodes close to the site of subcutaneous injection may be advantageous, while a systemic immune stimulation may not.

In general, compounds or compositions should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

Compositions according to various embodiments of the invention may be provided in a unit dosage form, or in a bulk form suitable for formulation or dilution at the point of use. Compositions according to various embodiments of the invention may be administered to a subject in a single-dose, or in several doses administered over time. Dosage schedules may be dependent on, for example, the subject's condition, age, gender, weight, route of administration, formulation, or general health. Dosage schedules may be calculated from measurements of adsorption, distribution, metabolism, excretion and toxicity in a subject, or may be extrapolated from measurements on an experimental animal, such as a rat or mouse, for use in a human subject. Optimization of dosage and treatment regimens are discussed in, for example, Goodman & Gilman's The Pharmacological Basis of Therapeutics 11^{th} edition. 2006. L L Brunton, editor. McGraw-Hill, New York, or Remington—The Science and Practice of Pharmacy, 21^{st} edition. Gennaro et al editors. Lippincott Williams & Wilkins Philadelphia.

A “vaccine” is a composition that includes materials that elicit a desired immune response. A desired immune response may include protection against infection by a Chlamydia spp. pathogen. For example, a desired immune response may include any value from between 10% to 100%, e.g., 10%, 20%, 30%, 40%, 50%, 60%, 70%, 80%, 90%, 100%, protection against infection by a Chlamydia spp. pathogen in a vaccinated animal when compared to a non-vaccinated animal.

An “immune response” may generally refer to a response of the adaptive immune system, such as a humoral response, and a cell-mediated response. The humoral response is the aspect of immunity that is mediated by secreted antibodies, produced in the cells of the B lymphocyte lineage (B cell). Secreted antibodies bind to antigens on the surfaces of invading microbes (such as viruses or bacteria), which flags them for destruction. Humoral immunity is used generally to refer to antibody production and the processes that accompany it, as well as the effector functions of antibodies, including Th2 cell activation and cytokine production, memory cell generation, opsonin promotion of phagocytosis, pathogen elimination and the like. A cell-mediated response may refer to an immune response that does not involve antibodies but rather involves the activation of macrophages, natural killer cells (NK), antigen-specific cytotoxic T-lymphocytes, and the release of various cytokines in response to an antigen. Cell-mediated immunity may generally refer to some Th cell activation, Tc cell activation and T-cell mediated responses.

Antigen presenting cells (APCs) such as dendritic cells (DCs) take up polypeptides and present epitopes of such polypeptides within the context of the DC MHC I and II complexes to other immune cells including CD4+ and CD8+ cells. An

‘MHC complex’ or ‘MHC receptor’ is a cell-surface receptor encoded by the major histocompatibility complex of a subject, with a role in antigen presentation for the immune system. MHC proteins may be found on several cell types, including antigen presenting cells (APCs) such as macrophages or dendritic cells (DCs), or other cells found in a mammal. Epitopes associated with MHC Class I may range from about 8-11 amino acids in length, while epitopes associated MHC Class II may be longer, ranging from about 9-25 amino acids in length.

Accordingly, an “immune response” includes, but is not limited to, one or more of the following responses in a mammal: induction of antibodies, B cells, T cells (including helper T cells, suppressor T cells, cytotoxic T cells, γδ T cells) directed specifically to the antigen(s) in a composition or vaccine, following administration of the composition or vaccine. An immune response to a composition or vaccine thus generally includes the development in the host mammal of a cellular and/or antibody-mediated response to the composition or vaccine of interest. In general, the immune response will result in prevention or reduction of infection by a Chlamydia spp. pathogen. In some embodiments, an immune response refers specifically to a cell-mediated response. In some embodiments, an immune response refers specifically to a cell-mediated response against a Chlamydia spp. pathogen.

Vaccines according to the disclosure may include the polypeptides and nucleic acid molecules described herein, or immunogenic fragments thereof, and may be administered using any form of administration known in the art or described herein.

An “immunogenic fragment” of a polypeptide or nucleic acid molecule refers to an epitope or amino acid or nucleotide sequence that elicits an immune response. The term “epitope” refers to an arrangement of amino acids in a protein or modifications thereon (for example glycosylation). The amino acids may be arranged in a linear fashion, such as a primary sequence of a protein, or may be a secondary or tertiary arrangement of amino acids in close proximity once a protein is partially or fully configured. Epitopes may be specifically bound by an antibody, antibody fragment, peptide, peptidomimetic or the like, or may be specifically bound by a ligand or held within an MHC I or MHC II complex.

Thus, an immunogenic fragment may include, without limitation, any portion of any of the sequences described herein, or a sequence substantially identical thereto, that includes one or more epitopes (the site recognized by a specific immune system cell, such as a T cell). For example, an immunogenic fragment may include, without limitation, peptides of any value between 6 and 60, or over 60, amino acids in length, e.g., peptides of any value between 10 and 20 amino acids in length, or between 20 and 40 amino acids in length, derived from any one or more of the sequences described herein. Such fragments may be identified using standard methods known to those of skill in the art, such as epitope mapping techniques or antigenicity or hydropathy plots using, for example, the Omiga version 1.0 program from Oxford Molecular Group (see, for example, U.S. Pat. No. 4,708,871)(76, 77, 81, 92, 73,). An epitope may have a range of sizes - for example a linear epitope may be as small as two amino acids, or may be larger, from about 3 amino acids to about 20 amino acids. In some embodiments, an epitope may be from about 5 amino acids to about 10 or about 15 amino acids in length. An epitope of secondary or tertiary arrangements of amino acids may encompass as few as two amino acids, or may be larger, from about 3 amino acids to about 20 amino acids. In some embodiments, a secondary or tertiary epitope may be from about 5 amino acids to about 10 or about 15 amino acids in proximity to some or others within the epitope.

In some embodiments, a vaccine includes a suitable carrier, such as an adjuvant, which is an agent that acts in a non-specific manner to increase the immune response to a specific antigen, or to a group of antigens, enabling the reduction of the quantity of antigen in any given vaccine dose, or the reduction of the frequency of dosage required to generate the desired immune response.

Exemplary adjuvants include, without limitation, aluminum hydroxide, alum, Alhydrogel™ (aluminum trihydrate) or other aluminum-comprising salts, virosomes, nucleic acids comprising CpG motifs such as CpG oligodeoxynucleotides (CpG-ODN), squalene, oils, MF59 (Novartis), LTK63 (Novartis), QS21, various saponins, virus-like particles, monomycolyl glycerol (MMG), monophosphoryl-lipid A (MPL)/trehalose dicorynomycolate, toll-like receptor agonists, copolymers such as polyoxypropylene and polyoxyethylene, AbISCO, ISCOM (AbISCO-100), montanide ISA 51, Montanide ISA 720+CpG, etc. or any combination thereof. In some embodiments, exemplary adjuvants include a cationic lipid delivery agent such as dimethyldioctadecylammonium Bromide (DDA) together with a modified mycobacterial cord factor trehalose 6,6′-dibehenate (TDB) (DDA/TDB), DDA/MMG or DDA/MPL or any combination thereof. Liposomes with or without incorporated MPL further been adsorbed to alum hydroxide may also be useful, see, for example U.S. Pat. Nos. 6,093,406 and 6,793,923 B2. In some embodiments, exemplary adjuvants include prokaryotic RNA. In some embodiments, exemplary adjuvants include those described in for example US Patent Publication 2006/0286128 In some embodiments, exemplary adjuvants include DDA/TDB, DDA/MMG or DDA/MPL and prokaryotic RNA.

In some embodiments, vaccine compositions include, without limitation, peptides or polypeptides from a combination of PmpG, PmpE, PmpF and PmpH and, optionally, MOMP, in combination with DDA/TDB, DDA/MMG or DDA/MPL and, optionally, prokaryotic RNA.

In some embodiments, compounds for use in the compositions and methods according to the disclosure include, without limitation, peptides or polypeptides from a combination of PmpG, PmpE, PmpF and TC0420 and, optionally, MOMP, in combination with DDA/TDB, DDA/MMG or DDA/MPL and, optionally, prokaryotic RNA.

In some embodiments, a composition as described herein may be used to inoculate a test subject, for example, an animal model of Chlamydia infection, such as a mouse. Methods of experimentally inoculating experimental animals are known in the art. For example, testing a Chlamydia spp. vaccine may involve infecting previously inoculated mice intranasally with an inoculum comprising an infectious Chlamydia strain, and assessing for development of pneumonia. An exemplary assay is described in, for example Tammiruusu et al 2007. Vaccine 25(2):283-290, or in Rey-Ladino et al 2005. Infection and Immunity 73:1568-1577. It is within the ability of one of skill in the art to make any minor modifications to adapt such an assay to a particular pathogen model.

In another example, testing a Chlamydia vaccine may involve serially inoculating female mice with a candidate T-cell antigen cloned and expressed as described above. A series of inoculations may comprise two, three or more serial inoculations. The candidate T-cell antigens may be combined with an adjuvant. About three weeks following the last inoculation in the series, mice may be treated subcutaneously with 2.5 mg Depo-Provera and one week later both naive and immunized mice may be infected intravaginally with Chlamydia. The course of infection may be followed by monitoring the number of organisms shed at 2 to 7 day intervals for 6 weeks. The amount of organism shed may be determined by counting Chlamydia inclusion formation in HeLa cells using appropriately diluted vaginal wash samples. Immunity may be measured by the reduction in the amount of organism shed in immunized mice compared to naive mice.

In some embodiments, the present disclosure also provides for a composition for inducing an immune response in a subject. Compositions according to various embodiments of the invention may be used as a vaccine, or in the preparation of a vaccine.

In another embodiment, a peptide or polypeptide as described herein may be used in the preparation of a medicament such as a vaccine composition, for the prevention or treatment of a Chlamydia infection. Treatment or treating includes prevention unless prevention is specifically excluded, as in alternative embodiments of the disclosure. Treatment or treating refers to fully or partially reducing severity of a Chlamydia infection and/or delaying onset of a Chlamydia infection, and/or reducing incidence of one or more symptoms or features of a Chlamydia infection, including reducing survival, growth, and/or spread of a Chlamydia spp., such as C. muridarum or C. trachomatis. In some embodiments, treatment includes inducing immunity in an animal subject. In alternative embodiments, treatment includes inducing cellular immunity in an animal subject. Treatment may be administered to a subject who does not exhibit signs of a disease, disorder, and/or condition (an asymptomatic subject), and/or to a subject who exhibits only early signs of a disease, disorder, and/or condition for the purpose of decreasing the risk of developing pathology associated with the disease, disorder, and/or condition. In some embodiments, treatment includes delivery of an immunogenic composition (e.g., a vaccine) to a subject.

The composition or medicament may be used for the prevention or treatment of a Chlamydia infection in a subject having, or suspected of having such an infection. In some embodiments, the composition or medicament may be used for the prevention or treatment of urogenital or ocular conditions. Urogenital conditions include without limitation urethritis, cervicitis, pharyngitis, proctitis, epididymitis, and prostatis. Ocular conditions include without limitation trachoma and conjunctivitis.

In some embodiments, the peptides or polypeptides described herein, alone or in combination, may be used to diagnose the presence of a Chlamydia infection in a subject for example even in an asymptomatic subject. Diagnosis may be determine T cell responses and may be performed using any technique described herein or known to the skilled person.

### Articles of Manufacture

Also provided is an article of manufacture, comprising packaging material and a composition comprising one or more peptides or polypeptides as provided herein. The composition includes a physiologically or pharmaceutically acceptable excipient, and may further include an adjuvant, a delivery agent, or an adjuvant and a delivery agent, and the packaging material may include a label which indicates the active ingredients of the composition (e.g. the peptide or polypeptide, adjuvant or delivery agent as present). The label may further include an intended use of the composition, for example as a therapeutic or prophylactic composition to be used in the manner described herein.

### Kits

In another embodiment, a kit for the preparation of a medicament, comprising a composition comprising one or more peptides as provided herein, along with instructions for its use is provided. The instructions may comprise a series of steps for the preparation of the medicament, the medicament being useful for inducing a therapeutic or prophylactic immune response in a subject to whom it is administered. The kit may further comprise instructions for use of the medicament in treatment for treatment, prevention or amelioration of one or more symptoms of a Chlamydia infection, and include, for example, dose concentrations, dose intervals, preferred administration methods or the like.

The present invention will be further illustrated in the following examples.

### EXAMPLES

### Materials and Methods

Chlamydia

C. muridarum mouse pneumonitis (MoPn) strain Nigg was grown in Hela 229 in Eagle's minimal essential medium (Invitrogen) supplemented with 10% FCS. Elementary bodies (EBs) were purified by discontinuous density gradient centrifugation as previously described (Caldwell, H. D., J. Kromhout, and J. Schachter. 1981. Purification and partial characterization of the major outer membrane protein of Chlamydia trachomatis. Infect Immun 31:1161-1176.). Purified EBs were aliquoted and stored at −80° C. in sucrose-phosphate-glutamic acid buffer and thawed immediately before use. The infectivity and the number of inclusion-forming units (IFU) of purified EBs was determined by immunostaining using anti-EB mouse polyclonal antibody followed by biotinylated anti-mouse IgG (Jackson ImmunoResearch Laboratories) and a diaminobenzidine (DAB) substrate (Vector Laboratories) (Yang, X., K. T. HayGlass, and R. C. Branham. 1996. Genetically determined differences in IL-10 and IFN-gamma responses correlate with clearance of Chlamydia trachomatis mouse pneumonitis to infection. J Immunol 156:4338-4344). The IFU for live EBs was calculated from the titers determined on original C. muridarum EB purified stocks as described above.

Mice

Female C57BL/6 or BALB/c mice (5 to 6 week old) were purchased from Charles River Canada and housed under pathogen-free conditions.

Isolation and Mass Spectrometric Identification of MHC-Binding Peptides using the Immunoproteomic Approach

The overall process for identification of candidate T-cell antigens for a Chlamydia vaccine used in this invention is shown schematically in FIG. 1 and provided in greater detail below.

DC Pulsing with Live EBs

DCs were generated as previously described (Inaba, K., M. Inaba, N. Romani, H. Aya, M. Deguchi, S. Ikehara, S. Muramatsu, and R. M. Steinman. 1992. Generation of large numbers of dendritic cells from mouse bone marrow cultures supplemented with granulocyte/macrophage colony-stimulating factor. J Exp Med 176:1693-1702). Briefly, bone marrow cells were isolated from the femurs or tibias of

BALB/c mice and cultured in Falcon petri dishes at 4×10⁷ cells in 50 ml DC medium. DC medium is Iscove's modified Dulbecco's medium (IMDM) supplemented with 10% FCS, 0.5 mM 2-ME, 4 mM L-glutamine, 50 μg/ml gentamicin, and 5% of culture supernatant of murine GM-CSF-transfected plasmacytoma X63-Ag8 and 5% of culture supernatant of murine IL-4 transfected plasmacytoma X63-Ag8 which contained 10 ng/ml GM-CSF and 10 ng/ml IL-4, respectively. At day 3, half of culture supernatants were removed and fresh DC medium was added. At day 5, non-adherent cells (purity of >50% CD11c+), designated bone marrow-derived dendritic cells (BM-DCs) were transferred to new dishes and cultured at 25×107 cells in 50 ml DC medium containing 25×107 IFU live EBs at 37° C. in 5% CO2 for 12 h. The cells pulsed with live EB were then harvested and stored in −80° C.

Identification of MHC Class II-Bound Peptides

We acquired 6×10⁹ BM-DCs pulsed with live EBs. The immunoproteomic approach to identify MHC class II-bound peptides from pulsed DCs involved multiple steps was previously described (Karunakaran, K. P., J. Rey-Ladino,

N. Stoynov, K. Berg, C. Shen, X. Jiang, B. R. Gabel, H. Yu, L. J. Foster, and R. C. Brunham. 2008. Immunoproteomic discovery of novel T cell antigens from the obligate intracellular pathogen Chlamydia. J Immunol 180:2459-2465). Briefly, the pulsed DCs were lysed and MHC class II (I-Ab) molecules were purified using allele-specific anti-MHC monoclonal antibody affinity columns. MHC class II molecules bound to the affinity column were then eluted and the MHC-bound peptides were separated from MHC molecules by acetic acid treatment and ultrafiltration through a 5-kDa cutoff membrane to remove high molecular mass material. The purified MHC-bound peptides were analyzed qualitatively using an LTQ-OrbitrapXL (Thermo Electron) on-line coupled to a nanoflow HPLC using a nanospray ionization source. This mass spectrometer is set to fragment the five most intense multiply-charged ions per cycle. Fragment spectra are extracted using DTAS up erCharge (http://msquant.sourceforge.net) and searched using the Mascot algorithm against a database comprised of the protein sequences from C. muridarum.

Statistical Analysis

Data were analyzed with the aid of the GraphPad Prism software program. The Kruskal-Wallis test was performed to analyze data for C. muridarum sheddings from multiple groups, and the Mann-Whitney U test was used to compare medians between pairs. P values of <0.05 were considered significant. Data are presented as means ±standard errors of the means (SEM).

Identification of Candidate T-cell Vaccine Antigens by Immunoproteomics (Isolation and mass spectrometric identification of MHC Binding Peptides)

Table 1 lists antigens identified by application of the immunoproteomic approach under slightly modified experimental conditions. In this case, bone-marrow derived dendritic cells (BM-DCs) were isolated from BALB/c mice (as opposed to the C57BL/6 strain) and were incubated with C. muridarum for 12 hours.

Table 2 lists T-cell antigens identified separately in two previous studies employing distinct experimental conditions.

TABLE 1
Chlamydia T cell antigens identified by immunoproteomic approach
after bone-marrow dendritic cells from BALB/c mice were infected
with Chlamydia for 12 hrs

| Chlamydia | | | | Chlamydia |
|---|---|---|---|---|
| muridarum | | | Abbre- | trachomatis |
| Locus# | Peptide sequence | Source protein | viation | Locus# |
| TC0264 | SPQVLTPNVIIPFKGDD | Polymorphic | PmpH | CT872 |
| (SEQ ID NO: 57) | (SEQ ID NO: 1) | membrane | | (SEQ ID NO: 58) |
| | | protein H | | |
| TC0895 | SMLIIPALGG | Nucleoside | YggV | CT606 |
| (SEQ ID NO: 59) | (SEQ ID NO: 2) | triphosphatase | | (SEQ ID NO: 60) |
| TC0839 | LAAAVMHADSGAILKEK | D-analyl-D- | DacC | CT551 |
| (SEQ ID NO: 61) | (SEQ ID NO: 3) | alanine | | (SEQ ID NO: 62) |
| | | carboxypeptidase | | |
| TC0825 | DDPEVIRAYIVPPKEP | Hypothetical | | CT538 |
| (SEQ ID NO: 63) | (SEQ ID NO: 4) | protein | | (SEQ ID NO: 64) |
| TC0755 | KIFSPAGLLSAFAKNGA | DNA repair | RecO | CT470 |
| (SEQ ID NO: 65) | (SEQ ID NO: 5) | protein | | (SEQ ID NO: 66) |
| TC0745 | DPVDMFQMTKIVSKH | SWIB (YM74) | | CT460 |
| (SEQ ID NO: 67) | (SEQ ID NO: 6) | complex protein | | (SEQ ID NO: 68) |
| TC0741 | KLEGIINNNNTPS | Translocated | Tarp | CT456 |
| (SEQ ID NO: 69) | (SEQ ID NO: 7) | actin-recruiting | | (SEQ ID NO: 70) |
| | | phosphoprotein | | |
| TC0021 | AVPRTSLIF | Exodeoxyribo- | RecD_2 | CT652 |
| (SEQ ID NO: 71) | (SEQ ID NO: 8) | nuclease V, | | (SEQ ID NO: 72) |
| | | alpha subunit | | |
| TC0372 | GGAEVILSRSHPEFVKQ | N utilization | NusA | CT097 |
| (SEQ ID NO: 73) | (SEQ ID NO: 9) | substance | | (SEQ ID NO: 74) |
| | | protein A | | |
| TC0285 | APILARLS | Hypothetical | | CT017 |
| (SEQ ID NO: 75) | (SEQ ID NO: 10) | protein | | (SEQ ID NO: 76) |

TABLE 2
MHC class II-bound C. muridarum-derived peptides and their
source proteins identified when murine bone marrow derived
dendritic cells from C57BL/6 mice were infected with
C. muridarum for either 12 or 24 hrs.

| Chlamydia | | | | Chlamydia |
|---|---|---|---|---|
| muridarum | | | Abbre- | trachomatis |
| Locus# | Peptide sequence | Source protein | viation | Locus# |
| TC0262 | AFHLFASPAANYIHTG | Polymorphic membrane | PmpF | CT870 |
| | (SEQ ID NO: 11) | protein F | | |
| TC0263 | NAKTVFLSNVASPIYVDPA | Polymorphic membrane | PmpG | CT871 |
| | (SEQ ID NO: 12) | protein G | | |
| | ASPIYVDPAAAGGQPPA | | | |
| | (SEQ ID NO: 13) | | | |
| TC0801 | VKGNEVFVSPAAHIIDRPG | Ribosomal protein L6 | RplF | CT514 |
| | (SEQ ID NO: 14) | | | |
| TC0508 | SPGQTNYAAAKAGIIGFS | 3-oxoacyl-(acyl carrier | FabG | CT237 |
| | (SEQ ID NO: 15) | protein) reductase | | |
| TC0707 | KLDGVSSPAVQESISE | Ani-anti-sigma factor | Aasf | CT424 |
| | (SEQ ID NO: 16) | | | |
| TC0079 | IGQEITEPLANTVIA | ATP dependent Clp | ClpP | CT706 |
| | (SEQ ID NO: 17) | protease, proteolytic | | |
| | | subunit | | |
| TC0792 | MTTVHAATATQSVVD | Glyceraldehyde 3- | Gap | CT505 |
| | (SEQ ID NO: 18) | phosphate | | |
| | | dehydrogenase | | |
| TC0420 | DLNVTGPKIQTDVD | Hypothetical protein | | CT143 |
| | (SEQ ID NO: 19) | | | |
| TC0518 | EGTKIPIGTPIAVFSTEQN | Pyruvate dehydrogenase | PdhC | CT247 |
| | (SEQ ID NO: 20) | | | |
| TC0884 | SVPSYVYYPSGNRAPVV | Thiol disulfide | DsbD | CT595 |
| | (SEQ ID NO: 21) | interchange protein | | |
| TC0654 | YDHIIVTPGANADIL | Oxidoreductase, DadA | | CT375 |
| | (SEQ ID NO: 22) | family | | |
| TC0190 | LPLMIVSSPKASESGAA | Metalloprotease, | | CT806 |
| | (SEQ ID NO: 23) | insulinase family | | |
| TC0721 | GANAIPVHCPIGAESQ | Translation elongation | FusA | CT437 |
| | (SEQ ID NO: 24) | factor G | | |
| | VFWLGSKINIIDTPG | | | |
| | (SEQ ID NO: 25) | | | |
| TC0050 | ISRALYTPVNSNQSVG | Translation elongation | Tsf | CT679 |
| | (SEQ ID NO: 26) | factor Ts | | |
| TC0596 | FEVQLISPVALEEGMR | Translation elongation | Tuf | CT322 |
| | (SEQ ID NO: 27) | factor Tu | | |
| | GDAAYIEKVRELMQ | | | |
| | (SEQ ID NO: 28) | | | |
| TC0261 | SRALYAQPMLAISEA | Polymorphic membrane | PmpE | CT869 |
| | (SEQ ID NO: 29) | protein E | | |
| TC0584 | KPAEEEAGSIVHNAREQ | V-type, ATP synthase | AtpE | CT310 |
| | (SEQ ID NO: 30) | subunit E | | |

In the first study (1^{st} set of eight antigens in Table 2), the T-cell antigens were identified as presented by MHC class II molecules when BM-DCs from C57BL/6 mice were infected with Chlamydia for 24 hrs (Karunakaran, K. P., J. Rey-Ladino, N. Stoynov, K. Berg, C. Shen, X. Jiang, B. R. Gabel, H. Yu, L. J. Foster, and R. C. Brunham. 2008. Immunoproteomic discovery of novel T cell antigens from the obligate intracellular pathogen Chlamydia. J Immunol 180:2459-2465). In the second study (remaining nine antigens in Table 2), these nine T-cell antigens were identified as presented by MHC class II molecules when BM-DCs derived from C57BL/6 mice were infected with Chlamydia for 12 hours (Yu H, Karunakaran K P, Kelly I, Shen C, Jiang to X, Foster L J, Brunham R C. Immunization with live and dead Chlamydia muridarum induces different levels of protective immunity in a murine genital tract model: correlation with MHC class II peptide presentation and multifunctional Th1 cells. J Immunol. 2011 March 15;186(6):3615-21. Epub 2011 February 4).

The immunoproteomic approach was also applied to identify 27 different C. trachomatis epitopes (Table 3) presented by MHC class II molecules after murine BM-DCs (C57BL/6) were infected for 12 hours with live C. trachomatis.

TABLE 3
MHC class II-bound C. trachomatis derived peptides and their source
proteins identified when murine (C57BL/6) bone marrow derived
dendritic cells were infected with live C. trachomatis for 12 hours
(10 overlapping proteins with C. muridarum are in bold).

| | Chlamydia | | Protein |
|---|---|---|---|
| | trachomatis | | Abbre- |
| Peptide | Locus# | Source Proteins | viation |
| KPAPKETPGAAEGAEAQTA | CT559 | Yop proteins translocation | CdsJ |
| SEQPSKENAEKQEENNEDA | | lipoprotein | |
| (SEQ ID NO: 31) | | | |
| GSVVFSGATVNSADFH | CT869 | Polymorphic membrane | PmpE |
| (SEQ ID NO: 32) | | protein E | |
| KLDGVSSPAVQESISESL | CT424 | Sigma Regulatory factor | RsbV |
| (SEQ ID NO: 33) | | | |
| VKGNEVFVTPAAHVVDRPG | CT514 | 50S ribosomal protein L6 | RI6 |
| (SEQ ID NO: 14) | | | |
| AEKGGGAIYAPTIDISTNGGS | CT872 | Polymorphic membrane | PmpH |
| (SEQ ID NO: 34) | | protein H | |
| YDHIIVTPGANADILPE | CT375 | Predicted D-Amino Acid | |
| (SEQ ID NO: 35) | | Dehydrogenase | |
| ISYDYSSGNAEASSHN | CT837 | Hypothetical protein CT837 | |
| (SEQ ID NO: 36) | | | |
| GSPGQTNYAAAKAGIIGFS | CT237 | 3-ketoacyl-(acyl-carrier- | FabG |
| (SEQ ID NO: 37) | | protein) reductase | |
| GPKGRHVVIDKSFGSPQVT | CT110 | Chaperonin GroEL1 | GroEL1 |
| KDGVT | | | |
| (SEQ ID NO: 38) | | | |
| GKLIVTNPKSDISFGG | CT144 | Hypothetical protein CT144 | |
| (SEQ ID NO: 39) | | | |
| SPKEAAIAAARASLSPEEKR | CT289 | Hypothetical protein CT289 | |
| (SEQ ID NO: 40) | | | |
| GTKTPIGTPIAVFSTEQ | CT247 | Dihydrolipoamide | PdhC |
| (SEQ ID NO: 41) | | acetyltransferase | |
| IPFAKPDANLSAED | CT619 | Hypothetical protein CT619 | |
| (SEQ ID NO: 42) | | | |
| ADVLLLSPKASVSPGG | CT561 | Type III secretion translocase | CdsL |
| (SEQ ID NO: 43) | | | |
| IFDTTTLNPTIAGAGDVK | CT681 | Major Outer Membrane | MOMP |
| (SEQ ID NO: 44) | | Protein | |
| DSTHGSFAPQATFSDG | CT505 | Glyceraldehyde-3- | GapA |
| (SEQ ID NO: 45) | | phosphate dehydrogenase | |
| KEGEEDTAESAANEEPKAEA | CT664 | FHA domain; homology to | |
| SQEEE | | adenylate cyclase | |
| (SEQ ID NO: 46) | | | |
| EERVVGQPFAIAAVSDS | CT113 | Clp Protease ATPase | ClpB |
| (SEQ ID NO: 47) | | | |
| TPVESTTPVAPEISVVNAK | CT759 | Muramidase (invasin repeat | NlpD |
| (SEQ ID NO: 48) | | family) | |
| YKLVYQNALSNFSGKK | CT045 | Leucyl aminopeptidase | PepA |
| (SEQ ID NO: 49) | | | |
| FDGEKASVGAPTVGNAVVKG | CT420 | 50S ribosomal protein L21 | Rl21 |
| (SEQ ID NO: 50) | | | |
| DLKVTGPTIHTDLD | CT143 | Hypothetical protein CT143 | |
| (SEQ ID NO: 51) | | 33 | |
| KAPQFGYPAVQNSADS | CT622 | CHLPN 76kDa Homolog | |
| (SEQ ID NO: 52) | | | |
| TPSAVNPLPNPEIDS | CT472 | Hypothetical protein CT472 | |
| (SEQ ID NO: 53) | | | |
| DAGVPIKAPVAGIAMG | CT842 | Polyribonucleotide | Pnp |
| (SEQ ID NO: 54) | | Nucleotidyltransferase | |
| QVFQLITQVTGRSG | CT778 | Primosome assembly protein | PriA |
| (SEQ ID NO: 55) | | | |
| AMANEAPIAFIANVAG | CT871 | Polymorphic membrane | PmpG |
| (SEQ ID NO: 56) | | protein G | |

Ten of these T-cell antigens were in common/overlapped (orthologous) to T-cell antigens presented by MHC class II molecules when C. muridarum was used to infect BM-DCs. These 10 orthologous proteins are shown in bold in Table 3 and separately in Table 4.

TABLE 4
T-cell Chlamydia antigens (MEC class II-bound peptides and
source proteins) presented in common between murine BM-DCs
infected by C. muridarum or C. trachomatis strains of
Chlamydia for 12 hrs.

| | Chlamydia | | Protein |
|---|---|---|---|
| | trachomatis | | Abbre- |
| Peptide | Locus# | Source Proteins | viation |
| GSVVFSGATVNSADFH | CT869 | Polymorphic membrane | PmpE |
| | | protein E | |
| KLDGVSSPAVQESISESL | CT424 | Sigma Regulatory factor | RsbV |
| VKGNEVFVTPAAHVVDRPG | CT514 | 50S ribosomal protein L6 | R16 |
| AEKGGGAIYAPTIDISTNGGS | CT872 | Polymorphic membrane | PmpH |
| | | protein H | |
| YDHIIVTPGANADILPE | CT375 | Predicted D-Amino Acid | |
| | | Dehydrogenase | |
| GSPGQTNYAAAKAGIIGFS | CT237 | 3-ketoacyl-(acyl-carrier- | FabG |
| | | protein) reductase | |
| GTKTPIGTPIAVFSTEQ | CT247 | Dihydrolipoamide | PdhC |
| | | acetyltransferase | |
| DSTHGSFAPQATFSDG | CT505 | Glyceraldehyde-3-phosphate | GapA |
| | | dehydrogenase | |
| DLKVTGPTIHTDLD | CT143 | Hypothetical protein CT143 | |
| AMANEAPIAFIANVAG | CT871 | Polymorphic membrane | PmpG |
| | | protein G | |

Evaluation of Protective Efficacy of Candidate T-Cell Vaccine Antigens Against Chlamydia Genital Infection in a Murine Model

Selected T-cell antigens identified by the immunoproteomic approach were evaluated for protective vaccine efficacy in a murine genital model of Chlamydia infection. These proteins (PmpG, PmpF, PmpE, PmpH, Rp1F, Aasf, RecO, Tarp, AtpE,

TC0420, TC0190, TC0825 and TCO285) have little or no sequence homology to human proteins and are present in Chlamydia or Chlamydia-related species. These proteins were also cloned, expressed and purified for subsequent immunization studies.

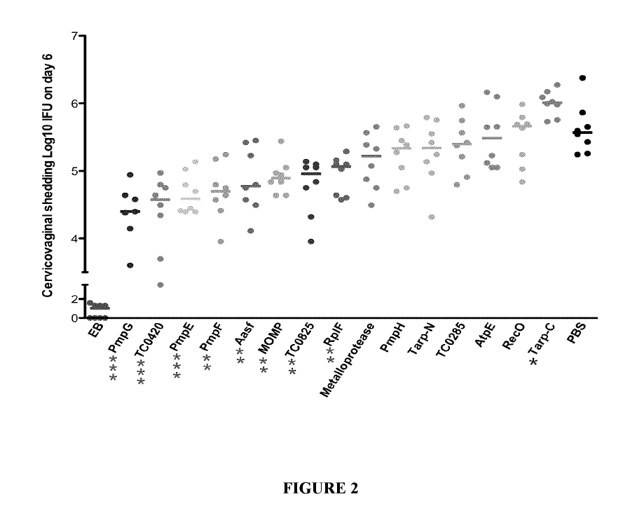

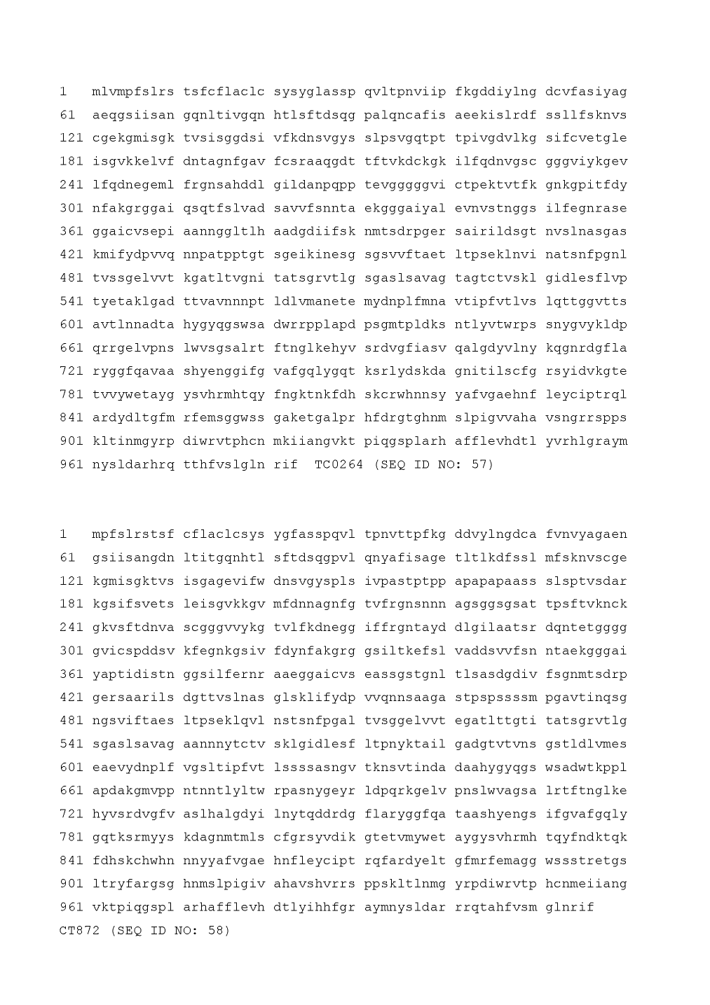

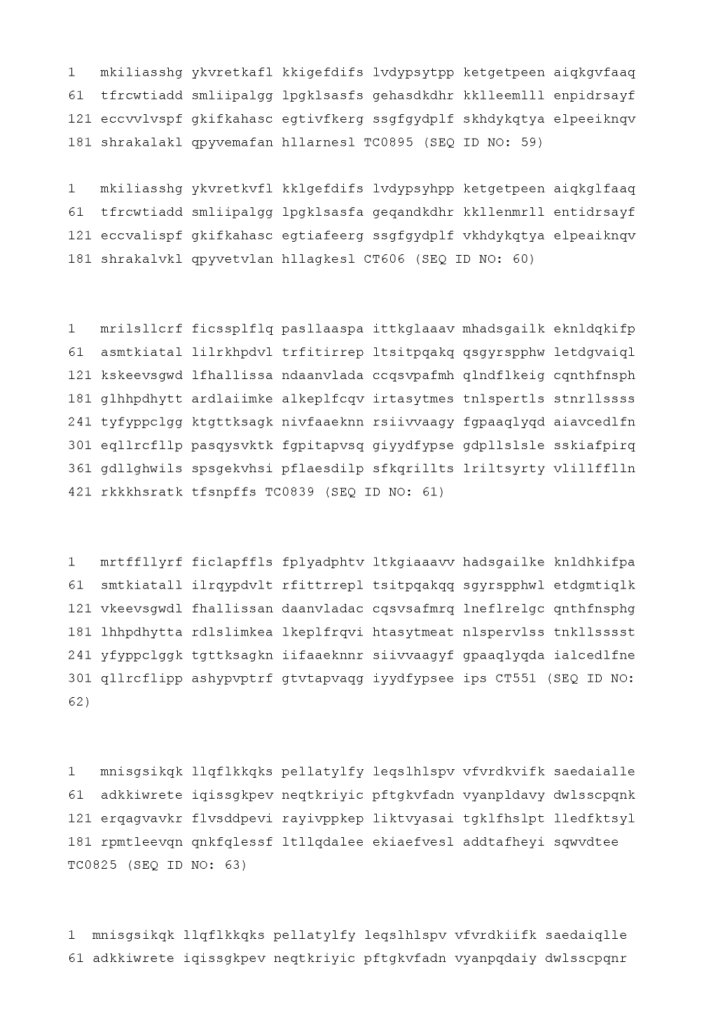

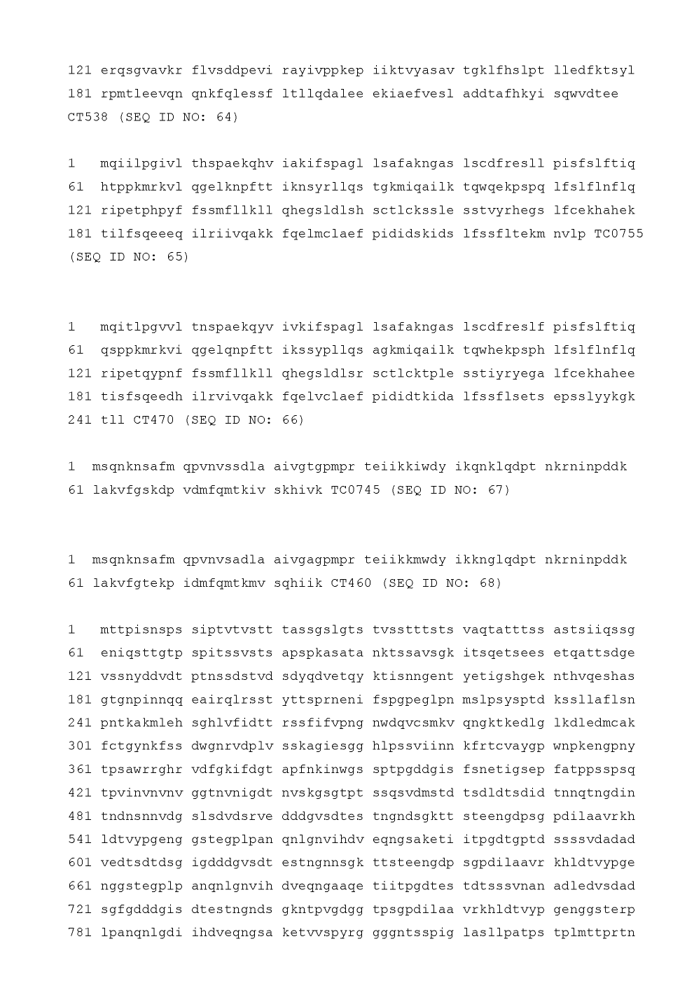

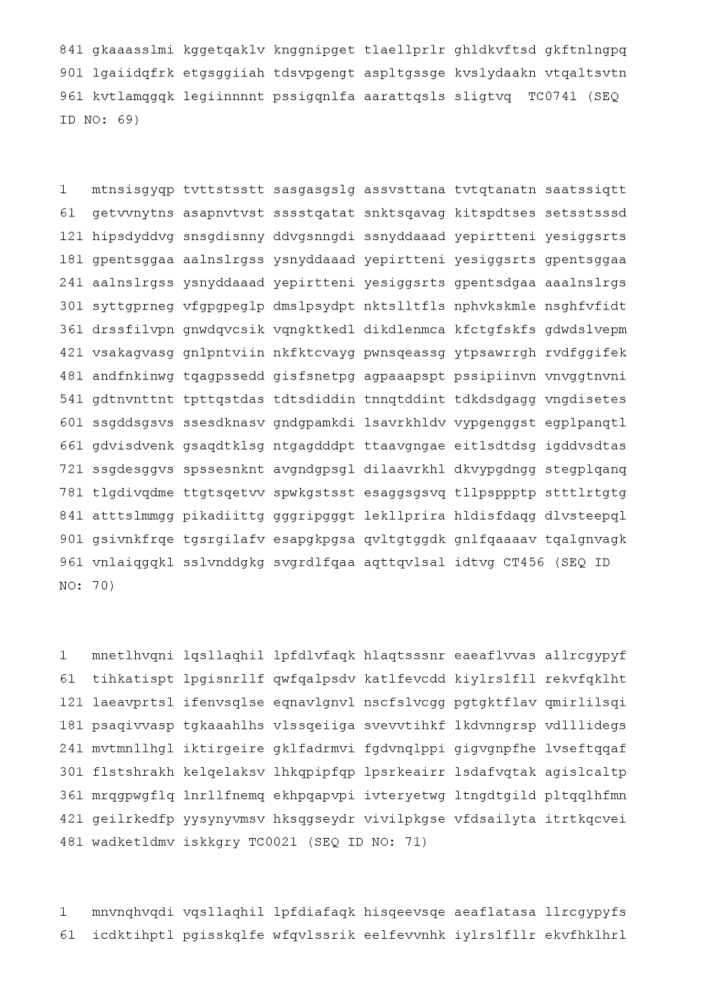

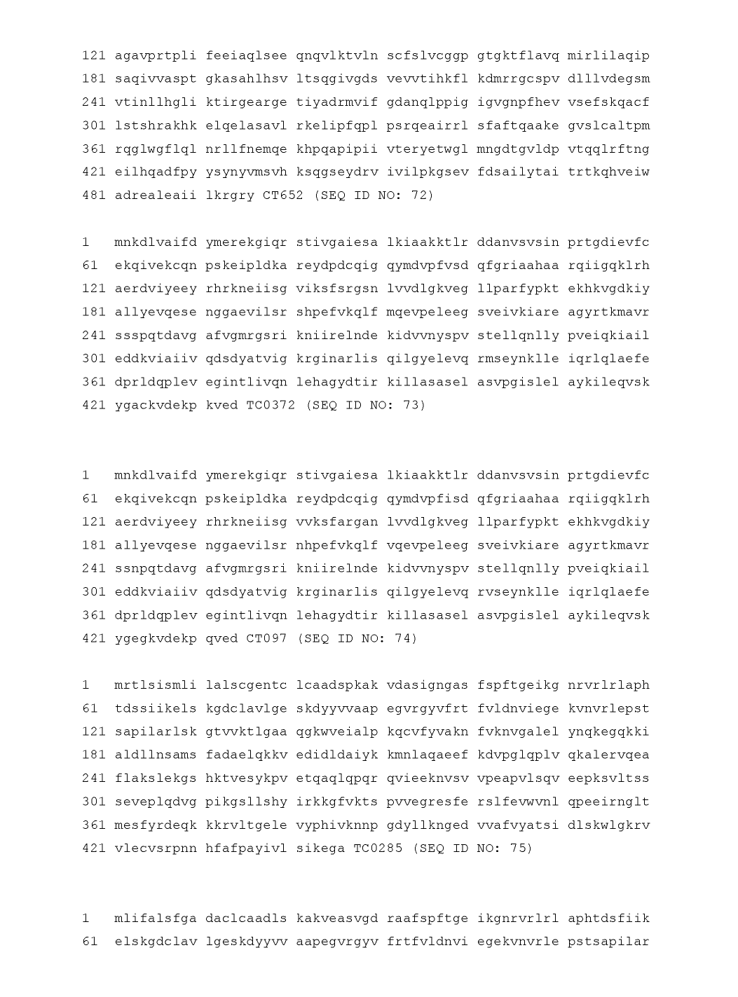

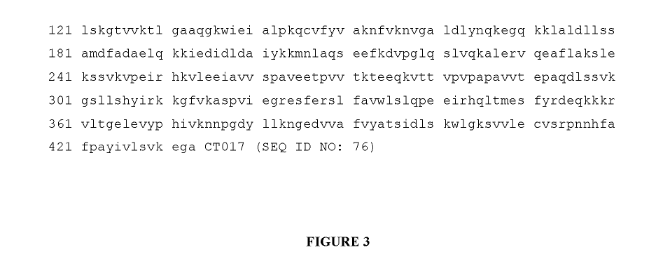

To evaluate whether these Chlamydia protein antigens were able to to protect mice against genital tract infection, mice were vaccinated with each recombinant protein (5 μg) and the reference antigen MOMP (5 μg) formulated with DDA/MPL adjuvant along with live EB as positive control and PBS as negative control. C57BL/6 mice were vaccinated three times with test antigens/controls with a 2-week interval. One week after the final immunization, the mice from each group were injected with Depo-Provera. One week after Depo-Provera treatment, the mice were infected intravaginally with 1500 IFU live C. muridarum EBs. Protection against intravaginal infection was assessed by isolation of Chlamydia from cervicovaginal washes and determination of the number of IFU recovered from each experimental group at day 6 post-infection (FIG. 2).

All citations are hereby incorporated by reference.

The present invention has been described with regard to one or more embodiments. However, it will be apparent to persons skilled in the art that a number of variations and modifications can be made without departing from the scope of the invention as defined in the claims.

1. An immunogenic composition comprising a polypeptide which comprises an amino acid sequence substantially identical to: SPQVLTPNVIIPFKGDD, SMLIIPALGG, LAAAVMHADSGAILKEK, DDPEVIRAYIVPPKEP, KIFSPAGLLSAFAKNGA, DPVDMFQMTKIVSKH, KLEGIINNNNTPS, AVPRTSLIF, GGAEVILSRSHPEFVKQ, APILARLS, or combinations thereof, together with a physiologically acceptable carrier.

2. The composition of claim 1 wherein the polypeptide comprises an amino acid sequence substantially identical to: Polymorphic membrane protein H (PmpH), Nucleoside triphosphatase (YggV), D-analyl-D-alanine carboxypeptidase (DacC), a hypothetical protein corresponding to locus tag CT538, DNA repair protein (RecO), SWIB (YM74) complex protein, Translocated actin-recruiting phosphoprotein (Tarp), Exodeoxyribonuclease V, alpha subunit (RecD_2), N utilization substance protein A (NusA), a hypothetical protein corresponding to locus tag CT017, or combinations thereof, together with a physiologically acceptable carrier.

3. The composition of claim 1 further comprising a polypeptide which comprises an amino acid sequence substantially identical to: AFHLFASPAANYIHTG, NAKTVFLSNVASPIYVDPA, ASPIYVDPAAAGGQPPA, VKGNEVFVSPAAHIIDRPG, SPGQTNYAAAKAGIIGFS, KLDGVSSPAVQESISE, IGQEITEPLANTVIA, MTTVHAATATQSVVD, DLNVTGPKIQTDVD, EGTKIPIGTPIAVFSTEQN, SVPSYVYYPSGNRAPVV, YDHIIVTPGANADIL, LPLMIVSSPKASESGAA, GANAIPVHCPIGAESQ, VFWLGSKINIIDTPG, ISRALYTPVNSNQSVG, FEVQLISPVALEEGMR, GDAAYIEKVRELMQ, SRALYAQPMLAISEA, or KPAEEEAGSIVHNAREQ, or combinations thereof.

4. The composition of claim 1 further comprising a polypeptide which comprises an amino acid sequence substantially identical to: Polymorphic membrane protein F (PmpF), Polymorphic membrane protein G (PmpG), Ribosomal protein L6 (RpIF), 3-oxoacyl-(acyl carrier protein) reductase (FabG), Anti-anti-sigma factor (Aasf), ATP dependent Clp protease, proteolytic subunit (CIpP), Glyceraldehyde 3-phosphate dehydrogenase (Gap), a hypothetical protein corresponding to locus tag CT143, Pyruvate dehydrogenase (PdhC), Thiol disulfide interchange protein (DsbD), Oxidoreductase, DadA family, Metalloprotease, insulinase family, Translation elongation factor G (FusA), Translation elongation factor Ts (Tsf), Translation elongation factor Tu (Tuf), Polymorphic membrane protein E (PmpE), V-type, ATP synthase subunit E (AtpE) , or combinations thereof.

5. The composition of claim 1 wherein the composition comprises PmpG, PmpE, PmpF and PmpH and, optionally, MOMP.

6. The composition of claim 1 wherein the composition comprises PmpG, PmpE, PmpF and TC0420 and, optionally, MOMP.

7. The composition of claim 1 further comprising an adjuvant.

8. The composition of claim 7 wherein the adjuvant is selected from DDA/TDB, DDA/MMG or DDA/MPL.

9. A method for eliciting an immune response against a Chlamydia spp., or component thereof, in an animal comprising administering to the animal an effective amount of the composition of claim 1, thereby eliciting an immune response in the animal.

10. The method of claim 9 wherein the immune response is a cellular immune response.

11. A method for treating or preventing infection by a Chlamydia spp. in an animal comprising administering to the animal an effective amount of the composition of claim 1, thereby treating or preventing infection by the Chlamydia spp. in the animal.

12. The method of claim 9 wherein the Chlamydia spp. is a Chlamydia trachomatis or a Chlamydia muridarum.

13. The method of claim 9 wherein the animal is a human.

14.-18. (canceled)

19. A method of diagnosing a Chlamydia spp. infection in an animal comprising determining the presence or absence of a T cell response to a polypeptide which comprises an amino acid sequence substantially identical to: SPQVLTPNVIIPFKGDD, SMLIIPALGG, LAAAVMHADSGAILKEK, DDPEVIRAYIVPPKEP, KIFSPAGLLSAFAKNGA, DPVDMFQMTKIVSKH, KLEGIINNNNTPS, AVPRTSLIF, GGAEVILSRSHPEFVKQ, or APILARLS, in a sample from the animal, wherein the presence of a T cell response indicates a Chlamydia spp. infection in the animal.

20. The method of claim 19 wherein the polypeptide comprises an amino acid sequence substantially identical to: Polymorphic membrane protein H (PmpH), Nucleoside triphosphatase (YggV), D-analyl-D-alanine carboxypeptidase (DacC), a hypothetical protein corresponding to locus tag CT538, DNA repair protein (RecO), SWIB (YM74) complex protein, Translocated actin-recruiting phosphoprotein (Tarp), Exodeoxyribonuclease V, alpha subunit (RecD_{—}2), N utilization substance protein A (NusA), a hypothetical protein corresponding to locus tag CT017.

21. The method of claim 19 wherein the sample consisting of vaginal fluid, vaginal tissue, vaginal washing, vaginal swab, urethral swab, urine, blood, serum, plasma, saliva, semen, urethral discharge, vaginal discharge, ocular fluid, ocular discharge or any combination thereof.

22. The method of claim 19 wherein the animal is human.

23. The method of claim 11 wherein the Chlamydia spp. is a Chlamydia trachomatis or a Chlamydia muridarum.

24. The method of claim 11 wherein the animal is a human.

25. The method of claim 19 wherein the Chlamydia spp. is a Chlamydia trachomatis or a Chlamydia muridarum.
